(19) United States
(12) Patent Application Publication
(10) Pub. No.: US 2015/0283072 A1
(43) Pub. Date: Oct. 8, 2015

(54) PRE-OPERATIVE BEVERAGES

(71) Applicant: Santé, LLC, Marion, MA (US)

(72) Inventor: Michael Popitz, Marion, MA (US)

(73) Assignee: SANTÉ, LLC, Marion, MA (US)

(21) Appl. No.: 14/656,183

(22) Filed: Mar. 12, 2015

Related U.S. Application Data
(60) Provisional application No. 61/955,986, filed on Mar. 20, 2014.

(51) Int. Cl. A61K 9/00; A23L 2/385; A23L 1/30; A61K 36/9064; A61K 36/66; A61K 36/48; A61K 36/73; A61K 36/9068; A61K 36/752; A61K 36/185; A61K 36/63; A61K 36/88; A61K 36/899; A61K 36/534; A61K 36/54; A61K 36/28; A61K 36/288; A23L 2/38

(52) U.S. Cl. CPC A61K 9/0095; A23L 2/38; A23L 2/385; A23L 1/3002; A61K 36/9064; A61K 36/66; A61K 36/48; A61K 36/73; A61K 36/9068; A61K 36/752; A61K 36/185; A61K 36/63; A61K 36/88; A61K 36/899; A61K 36/534; A61K 36/54; A61K 36/28; A61K 36/288; A23V 2002/00

(57) ABSTRACT

The invention provides a formulated beverage composition that can be administered to a patient, prior to anesthesia, sedation and/or surgical or dental operation, to reduce the risk of aspiration pneumonia while simultaneously improving the well-being of the patient. The beverage composition is designed to be taken orally, by a patient, within a specified short period of time prior to administration of an anesthesia and/or sedative/analgesic. Further described herein is the method of using the beverage composition. The method may include providing a written label of instructions affixed to the beverage container which instructions positively direct the patient's ingestion of the beverage composition.

### CROSS-REFERENCE TO RELATED APPLICATIONS

This application claims priority to and the benefit of the filing date under 35 USC 119(e) to U.S. provisional application 61/955,986, filed on Mar. 20, 2014, the entire content of which is incorporated herein by reference.

### BACKGROUND OF THE INVENTION

While conscious and in the erect position, the lower esophageal sphincter (LES) and laryngeal closing reflex of a healthy person prevent regurgitation and aspiration of stomach contents. Administration of anesthesia and/or sedative/analgesic drugs often compromises many natural reflexes, including those reflexes that help protect one's airway from such regurgitation and aspiration. Upon induction of and emergence from anesthesia, as well as intravenous sedation, a patient is at greatest risk for aspiration of gastric contents, particularly because the patient is usually supine, with no airway protection in place. One study has suggested that as many as 20% of patients given general anesthesia for surgery suffer at least some regurgitation and subsequent aspiration. See, Harris et al., Can. Anaesth. Soc. J. 31:599 (1984).

More than fifty years ago, Mendelson thoroughly described, for the first time, the symptoms and sequelae of aspiration of stomach contents into the lungs, now known generally as aspiration pneumonia (Mendelson, Am. J. Obstet. Gynecol. 52:191, 1946). Mendelson's observations, which were made of pregnant women anesthetized during labor, led him to describe two types of aspiration: 1) aspiration of solid gastric material, resulting in blockage of the patient's airway and risk of subsequent suffocation and/or pneumonia; and 2) aspiration of liquid gastric material, resulting in pneumonia and/or other asthma-like symptoms. Aspiration pneumonia, also sometimes referred to as Mendelson's Syndrome, was reproduced experimentally by injecting human acidic vomitus into rabbit tracheas. However, injection of neutralized vomitus caused no such symptoms. It is observations such as these that have led researchers to the conclusion that the acidity of the material aspirated from the stomach is the primary causative factor in aspiration pneumonia. See, for example, Kinni et al., J. Oral Maxillofac. Surg. 44:378-384, 1986; and Mendelson, Am. J. Obstet. Gynecol. 52:191, 1946.

While the fully anesthetized patient is at greatest risk of developing aspiration pneumonia, patients subjected to local anesthesia and/or sedative-analgesic medications are also at risk, particularly as the sedation nears the level of hypnosis, hence non-conscious sedation. To emphasize the importance of the proper monitoring of patients given sedative and/or analgesic medications prior to diagnostic or therapeutic procedures, the American Society of Anesthesiologists (ASA) has eliminated use of the term “conscious sedation” from its practice guidelines and instead refers to “sedation/analgesia.” In those guidelines, the ASA recommends that all patients receiving such medications be monitored by a designated individual who is primarily responsible for administration of the sedative and analgesic drugs and for subsequently monitoring the patient's vital signs, which person should not participate in performing the medical procedure. See “The Changing Role of Monitored Anesthesia Care in the Ambulatory Setting,” Rego et al., Anesth Analg. 85:1020-36, 1997, referring at page 1021 to “Task Force on Sedation and Analgesia by Non-Anesthesiologists. Practice guidelines for sedation and analgesia by non-anesthesiologists: A report by the American Society of Anesthesiologists Task Force on Sedation and Analgesia by Non-Anesthesiologists,” Anesthesiology 84:459-71, 1996. Thus, as used herein, references to the pre-anesthetized patient are intended to refer likewise to those patients scheduled to undergo local anesthesia and/or to be medicated with sedative-analgesic drugs for the purposes of performing a diagnostic and/or therapeutic procedure, as well as those destined to be anesthetized.

Efforts to eliminate aspiration pneumonia in the anesthetized/sedated patient have focused on both neutralizing the pre-operative stomach contents and reducing the residual gastric volume (RGV) present at induction of anesthesia and/or sedation/analgesia. To this end, guidelines were established for the pre-operative treatment of patients, which sought to reduce the risk of aspiration pneumonia by requiring the patient to abstain from all food and all but clear liquids for at least about 12 hours prior to induction and from all liquids for at least eight hours prior to induction. Traditionally, these guidelines have been implemented by a) requiring all patients to fast from midnight the day before their procedure (referred to as nulla per os—nothing by mouth—or the NPO guidelines); b) alkalinization and/or emptying of the stomach just prior to administration of anesthesia/sedation; c) particular vigilance for aspiration during induction of and emergence from anesthesia/sedation; and d) the use of tracheal intubation, during general anesthesia, to protect the airway and lungs from such regurgitation should it occur. These guidelines, though generally used as much as a century ago, still define the standard of care for the pre-operative patient and have seen little variation in all these years of use.

Until about twenty years ago, it was a relatively simple matter to enforce and monitor the NPO guidelines. Patients typically spent at least the night before surgery in the hospital and thus could be denied food and drink after midnight regardless of whether their surgery was scheduled for 8:00 a.m. the next morning or 4:00 p.m. the next afternoon. Further, these patients could be given intravenous hydration and/or glucose loading solutions to help alleviate the dry mouth, hunger, headaches and nausea frequently associated with fasting, not to mention the irritability those symptoms produce. Thus, compliance with these guidelines was rarely, if ever, a problem.

Today, however, most surgeries are performed on an outpatient basis, partly in response to technological and pharmacological advancements and partly in response to economic pressures, such as those brought about by managed care. It was estimated that, by the year 2000, 75% of all surgical procedures, more than 37 million operations per year, would be performed on an outpatient basis. This means that increasingly large numbers of patients will be relied upon to follow the pre-operative, NPO guidelines on their own without medical supervision. Prior to surgery, or other diagnostic or therapeutic procedure requiring anesthesia/sedation, each patient is asked whether he/she has fasted as instructed. Not only must the medical provider rely on the patient to honestly answer such questions, but, depending upon how the question is posed, the medical provider must also assume that the patient understood what it was he/she was supposed to not do.

A recent survey of patients at an out-patient facility revealed that although patients were given specific guidelines permitting them to ingest clear liquids up to 4 to 6 hours prior to their scheduled time for surgery, the majority of patients opted to fast from midnight, either out of concern about potential aspiration or because of misinterpretation of the instructions. Patients failed to follow the prescribed guidelines could have their surgeries cancelled or postponed, at significant expense to the facility and inconvenience to the patient, the medical providers and the medical staff. The patients' explanations for breaking the fast typically included forgetfulness, overwhelming thirst or hunger, and simple misunderstandings of the guidelines such as, what was meant by “clear liquids.” In addition to admitted violations, however, it must be assumed that some number of patients violated the guidelines but did not admit to doing so, and that still other patients failed to follow the guidelines, but did not realize that they had done so. Thus, the total number of patients that either intentionally or unintentionally violated the NPO guidelines would be expected to be much higher than that actually reported. This makes those patients be at even higher risk for peri-operative aspiration. Further, diabetic patients and parturients have delayed gastric emptying, and/or gastroparesis, a lazy stomach, further complicating the potential risks to these patients.

On the other hand, however, for the majority of the patients who appear to be compliant with the NPO guidelines, many of them complain of headaches, hunger, thirst, irritability, lightheadedness, and similar symptoms associated with fasting for long periods of time. This forced fasting prior to surgery almost always dehydrates patients. Their metabolic needs are also deranged due to this archaic fasting rule. Their feelings of normalcy and well-being are significantly diminished. They arrive to the preoperative area in physical and mental chaos due to the NPO after midnight rule.

Over the past 5-6 years, however, these NPO guidelines have been slowly loosened or liberalized, especially for ambulatory and pediatric surgical patients. Many surgical centers permit patients to have clear liquids up until 4-6 hours prior to coming to the surgical center for their scheduled procedures. Clear liquids are emptied from the stomach in approximately one hour. Currently, the clear liquids permitted the patients by the surgical centers include black tea or coffee, apple juice or water. These beverages must be non-carbonated and non-particulate (e.g., not composed of distinct particles, or particles of negligible sizes).

Overall, the above described experiences validate two conclusions of a recent larger scale survey concerning the NPO guidelines: (1) that long fasting prior to elective operation is not only uncomfortable for the patient but has detrimental effects, including causing thirst, hunger, irritability, noncompliance and resentment in adult patients, and (2) that confusion exists among physicians as to which “clear liquids” are appropriate prior to surgery, so it is no wonder that patients are not be able to identify the appropriate beverages. See, Green et al., Anesth. Anal. 83:123-8, 1996. Thus, it is clear that compliance with the NPO guidelines presents a serious problem with respect to modern day surgical procedures, which are preformed primarily on an outpatient basis.

In addition to issues regarding compliance with the NPO guidelines, the suitability of the guidelines for their purpose has recently been questioned. It has generally been accepted among those of skill in the art that a patient having a residual gastric volume (RGV) of about 25 ml or more (0.4 ml per kg body weight) and/or a gastric pH of about 2.5 or less, at the time of surgery, is at greatest risk of aspiration pneumonia. It is for this reason that the NPO guidelines have strictly required that patients fast for long periods of time prior to induction in order, presumably, to reduce the RGV and to increase or stabilize the pH of the patients' gastric contents. However, studies have shown that as many as 45% of elective surgery patients, despite having fasted as required, have RGV and/or pH values beyond these critical values and are therefore at significant risk for aspiration pneumonia. See Hutchinson et al., Anaesth. Intens. Care 3:198, 1975; Newson, Anaesth. Intens. Care 5:214, 1977, and Coobs et al., Anesth. Anal. 58:183, 1979. Furthermore, recent studies have suggested that prolonged fasting may actually increase gastric volume and acidity (that is, decrease pH), proving to be harmful to the pre-operative patient rather than protective. Scan et al., Can. Med. Assoc. J. 141:1151-4, 1989; Splinter et al., Can. J. Anaesth. 37:36-9, 1990; Sandbar et al., Anesth. 71:327-30, 1989; Hutchinson et al., Can. J. Anaesth. 35:12-5, 1988. In fact, for more than four decades, it has been known that normal adults and children, ingesting clear liquid volumes of 150 to 300 mL, are apparently able to empty their stomachs in 10 to 20 minutes, making the 8 hour fasting period normally required by the NPO guidelines seem quite extreme. See, Hunt, J. of Physiology (London) 132:267-88, 1956.

Despite the forgoing evidence that adherence to the NPO fasting guidelines does not sufficiently reduce either or both of the defined risks of aspiration pneumonia, most hospitals, clinics, and doctors adhere strictly to the guidelines.

Thus, what is needed is a new beverage composition and treatment method that will both address the need to reduce the acidity and volume of the stomach contents of the pre-operative patient and address the need to increase patient compliance with the required pre-operative treatment, whatever it may be.

Surgical patients also have many other iatrogenic significant handicaps prior to a surgical procedure. In some of these patients, pre-operative handicaps are similar to those anyone would have undergoing a stressful, important situation such as an important public speech, a large presentation, an important social event (such as a wedding ceremony), delivery of an infant (for both parents), and nonsurgical procedural events such as a dental procedure, cardiac catheterization or GI endoscopy.

A surgical patient may have an increased requirement for protection against nausea and emesis (both during the induction of an anesthetic, during the procedure and postoperatively, especially with narcotics given for pain relief), decreased immunity, deranged clotting mechanisms, lethargy and asthenia, anemia from loss of blood, and decreased function of the GI system. However, all these situations tend to leave the patient or the individual about to experience the stressful situation with increased anxiety, dehydration, and hunger, with a lack of metabolic nutritional substrate for many hours prior to a severe physical and emotional stress.

Thus, like pre-operative preparation, what is also needed is a beverage composition that better prepares the individual about to experience the stressful condition or event as well as to permit a generalized feeling of well-being.

Finally, a significant number of patients are those with neurologic impairment and or gastrointestinal impairment that also make them at high risk for aspiration. This population requires extra precaution during any anesthetic for multiple reasons, but particularly regarding their increased risk.

### SUMMARY OF THE INVENTION

Prolonged fasting prior to surgery usually lead to many negative ramifications, including dehydration, increased stress and patient discomfort, difficulty starting an intravenous line, electrolyte and carbohydrate imbalances, etc.

Thus one aspect of the invention provides an aqueous-based beverage composition having a neutral or near neutral pH (e.g., about pH 5.5-8, or about 6-8, or about 5.5-6.5, or about 6.5-7.5, or about 6, or about 7), wherein the beverage hydrates the patient towards euvolemia (versus the current pre-operative norm of hypovolemia); induces forward normal peristaltic action of the gastrointestinal (GI) tract (versus the common norm seen in today's patient preoperatively with stomach butterflies, nausea, hunger pains, and aerophagia); and/or induces a feeling of well-being and decreased anxiety.

The subject beverage composition decreases the risk of aspiration pneumonitis while it simultaneously improves the well-being of the patient. In contrast to the few products on the market or proposed products such as CLEARFAST®, the beverage composition of the invention does not load the patient with carbohydrates. A related beverage, however, may contain carbohydrates for non-diabetics with a variation also for diabetic patients.

By using the proper mixture and balance of water without carbonization, and salts and minerals consistent with their natural human intravascular concentrations, along with certain herbs, all blended into a pre-operative beverage, one could preoperatively or pre-stressful procedure or event, prepare any patient properly and safely.

Thus one aspect of the invention provides an aqueous beverage composition having a substantially neutral pH (e.g., about 6-8, or about 5.5-6.5, or about 6.5-7.5, or about 6, or about 7) and having substantially no calories from carbohydrates, wherein the beverage, upon consumption by, or administration to a subject, hydrates the subject towards euvolemia.

In a related aspect, the invention provides an aqueous beverage composition having a substantially neutral pH (e.g., about 6-8, or about 5.5-6.5, or about 6.5-7.5, or about 6, or about 7) and having substantially no calories from carbohydrates, wherein the beverage, upon consumption by, or administration to a subject, induces forward propulsion of the contents of the gastrointestinal (GI) tract of the subject by, for example, stimulation of normal peristaltic action.

In another related aspect, the invention provides an aqueous beverage composition having a substantially neutral pH (e.g., about 6-8, or about 5.5-6.5, or about 6.5-7.5, or about 6, or about 7) and having substantially no calories from carbohydrates, wherein the beverage, upon administration to a subject, induces a feeling of well-being and decreased anxiety in the subject.

In yet another related aspect, the invention provides an aqueous beverage composition having a substantially neutral pH (e.g., about 6-8, about 5.5-6.5, about 6.5-7.5, about 6, or about 7) and having substantially no calories from carbohydrates, wherein the beverage, upon administration to a subject, decreases sympathetic nervous system (SNS) function or activity in the subject, and/or increases parasympathetic nervous system (PSNS) function/activity.

In still another related aspect, the invention provides an aqueous beverage composition having a substantially neutral pH (e.g., about 6-8, or about 5.5-6.5, or about 6.5-7.5, or about 6, or about 7) and having substantially no calories from carbohydrates, wherein the beverage, upon administration to a pre-operative patient, decreases the risk of aspiration pneumonitis while simultaneously improves the well-being of the patient.

In certain embodiments, the subject is undergoing pre-operative preparation, and the beverage, upon administration to the subject, decreases the risk of aspiration pneumonitis while simultaneously improves the well-being of the patient.

In certain embodiments, the subject is about to experience a stressful condition or an important event. For example, the stressful condition and/or an important event may include: a public speech or presentation, a test/exam, a social event (wedding ceremony, attending sports event, etc.), a non-surgical procedure (e.g., a dental procedure, cardiac catheterization, or GI endoscopy).

In certain embodiments, the neutral pH is about 6-8, about 6.5-7.5, about 6, or about 7. In certain embodiments, the beverage maintains substantially neutral pH (e.g., about 6-8, about 5.5-6.5, about 6.5-7.5, about 6, or about 7) in case of aspiration of stomach contents.

In certain embodiments, the total calories are no more than about 150 Cal/serving, no more than about 100 Cal/serving, no more than about 50 Cal/serving, no more than about 20 Cal/serving.

In certain embodiments, the beverage is non-particulate. For example, the beverage may be non-particulate, which is beneficial in case of aspiration of stomach contents.

In certain embodiments, the beverage has a total salt concentration equivalent to or within 5%, 10%, or 15% of that of the intravascular fluid in said subject or patient.

In certain embodiments, the beverage has a total salt concentration of about 140 mEQ (e.g., about 100 mEQ, about 110 mEQ, about 120 mEQ, about 130 mEQ, about 150 mEQ, about 160 mEQ, about 170 mEQ, etc.).

In certain embodiments, the beverage comprises a first herbal composition that induces normal peristalsis with forward propulsion of the contents of the gastrointestinal (GI) tract of the subject. For example, the first herbal composition may comprise: Chinese angelica, cardamom (e.g., cardamom seed), ginger (e.g., ginger root extract), peppermint, fennel, cinnamon, cannabis, caraway, tangerine peel, slippery elm, plantain seeds and husks, senna, or mixtures thereof.

Additional first herbal composition may comprise: Clove bud, meadow sweet, goldthread root, hops, cannabis, alfalfa herb, anise seed, Hysop, Chamomile flower, slippery elm, wild corn poppy, Raspberry leaf, red clover, or spearmint, plantain, or a mixture of two or more thereof.

Tangerine peel, peppermint, cinnamon all have dual roles in being flavorants as well as pro-peristaltic agents.
Lotus corniculatus—sedative
Catnip Leaf—sedative

In certain embodiments, the beverage comprises a second herbal composition that induces a feeling of well-being and decreased anxiety in the subject. For example, the second herbal composition comprises: chamomile, lemon balm, American Valerian root, calendula, meadowsweet, lemon verbena, lemon grass, hops, passion flowers, valerian, mimosa flowers, mullein flower, hysop (leaf), basil (leaf), linden, catnip (leaf), Lotus corniculatus, bitter orange leaf or flowers, Jasmin, lavender, dandelion root, or California poppy, or mixtures thereof.

In certain embodiments, the ratio or concentration of the sedative and relaxing herbs can be manipulated/increased accordingly, in order to promote a more relaxed feeling or even sleepiness in certain subjects, e.g., for patients that suffer from insomnia and/or have GERD (gastroesophageal reflux disease).

In certain embodiments, the beverage comprises a third herbal composition that decreases sympathetic nervous system (SNS) function or activity (or “anti-SNS” for short) in the subject. For example, the third herbal composition comprises: Jasmin, lavender, hops, valerian, chamomile, passion flower, lemon balm, linden, California poppy, dandelion root and spearmint, tangerine, or mixtures thereof.

This mélange preferably also include an herbal flavorant that, in addition to its naturally palatable flavor and color, also has inherent effects of anxiolysis. For example, it might include bitter orange peel, leaf or flowers. That is, the flavorant may comprise one or more of: orange peel, tangerine, lemongrass, poppy, spearmint, lemon balm, cinnamon, California poppy, meadowsweet, Raspberry Leaf, Caraway, and lemon, etc.

In certain embodiments, the beverage comprises an herbal composition that stimulates the immune system. For example, the herbal composition may comprise: Echinacea spp., ginseng (Panax and/or Siberian), or mixtures thereof.

In certain embodiments, the first, second, or third herbal composition is US FDA-approved for administration to human.

In certain embodiments, the beverage reduces fears, decreases pre-operative jitters or butterfly stomachs, reduces acidity of the stomach contents, reduces an incidence of nausea with an empty stomach or a feeling of pre-operative parchedness, and/or reduces pre-operative headaches from stress and migraines.

In certain embodiments, the ratio or amount of the second herbal composition is adjusted to induce a more relaxed feeling or even sleepiness in the subject. In certain embodiments, the subject has insomnia and/or GERD (gastroesophageal reflux disease).

Another aspect of the invention provides a concentrate or an essentially dry mixture, suitable for use in forming any one of the subject aqueous beverages, comprising an admixture of materials in amounts and ratios such that, when constituted with water, said aqueous beverage is formed. The water can be hot, warm, or at around room temperature (e.g., 20-25° C.), or preferably not cold. For good absorption, the beverage can be ingested warm as a tea.

In certain embodiments, the concentrate or dry mixture of the invention substantially corresponds to any one of the examples illustrated herein (i.e., Examples 1-9). For example, a subject concentrate or dry mixture may have exactly the same amount or concentration of the ingredients listed in one of the Examples. A subject concentrate or dry mixture may also be based on any one of the Examples, but differ in that one, two, three, or four of the ingredients has increased or decreased amount or proportion, e.g., 5%, 10%, 15%, 20%, 25%, 30%, 35%, 40%, 50%, 60%, 70%, 80%, 90%, 100% more or less than the indicated amount or proportion.

Another aspect of the invention provides a method of preparing a patient for induction of anesthesia for surgery, the method comprising instructing the patient to ingest, beginning on midnight prior to the day said surgery or anesthesia is scheduled for the patient, an aqueous beverage composition of the invention ad lib.

In certain embodiments, the method further comprises instructing the patient to refrain from ingesting the aqueous beverage composition about 2-3 hours before said induction, about 1-2 hours before said induction, about 30-60 minutes before said induction, or about 15-30 minutes before said induction.

Another aspect of the invention provides a method of preparing a patient for induction of anesthesia for surgery, the method comprising causing the patient to ingest, beginning on midnight prior to the day said surgery or anesthesia is scheduled for the patient, any one of the subject aqueous beverage compositions ad lib.

In certain embodiments, the method further comprises causing the patient to refrain from ingesting the aqueous beverage composition about 2-3 hours before said induction, about 1-2 hours before said induction, about 30-60 minutes before said induction, or about 15-30 minutes before said induction.

Another aspect of the invention provides a method of preparing a subject for experiencing a stressful condition or participating in a social event, the method comprising instructing the subject to ingest, prior to experiencing the stressful condition or participating in the social event, any one of the subject aqueous beverage compositions ad lib.

In certain embodiments, the subject aqueous beverage composition is ingested at least about 5 min., 10 min., 15 min., 20 min., 30 min., 45 min., 1 hr., 2 hrs., 3 hrs., 4 hrs., 5 hrs., 6 hrs., 7 hrs., 8 hrs., 9 hrs., 10 hrs., 11 hrs., 12 hrs., 15 hrs., 18 hrs., 20 hrs., or 24 hrs. prior to experiencing the stressful condition or participating in the social event.

It should be understood that any embodiments described herein, including those described only in one section of the application or under one aspect of the invention, are also applicable to any other aspects of the invention, unless specifically prohibited or disclaimed.

### DETAILED DESCRIPTION OF THE INVENTION

For many centuries in many cultures, many herbal preparations have been available to render aid to the society, in increasing the production of red blood cells to fight anemia, to increase the strength of the immune system, to render assistance in the battles against nausea and emesis, and against lethargy and asthenia, to pre-hydrate or simply hydrate a patient, to increase the rate of wound healing, to alleviate anxiety, to improve the function of the digestive tract, to decrease post operative pain, to increase post-operative mental alertness, to stimulate immune system, as well as to permit a generalized feeling of well-being.

Thus one aspect of the invention provides a palatable, clear, neutral pH and non-particulate non-carbonated liquid beverage composition to patients or pre-stressor clients. The beverage composition is essentially based upon a blend of water and one or more herbal contents that will prepare the patients or clients for a significant stress, such as surgery or dental procedure, as well as allowing them to recover more smoothly due to the combined effects of the contents of the beverage.

For example, starting on midnight prior to any surgical/dental procedure or anesthetic for which the patient is scheduled, he or she should be given or should be permitted to drink the pre-operative beverage composition. They should ingest this beverage ad lib as their normal physiological needs require them to.

This beverage should be based upon a pH neutral (e.g., about 6-8, or about 5.5-6.5, or about 6.5-7.5, or about 6, or about 7) combination of water, salts and minerals, preferably having salts (such as sodium chloride or other physiologically compatible salts) that maintain similar salt concentration as intravascular fluid. For example, in terms of sodium chloride, the total concentration maybe about 140 mEQ, or within 5% thereof.

The subject beverage may also contain, depending on the intended purpose (such as those indicated after each ingredient/herbal composition), one or more such ingredients as:
Echinacea—to improve immune system
Ginseng—to decrease sense of weakness and exhaustion, anti-stress
Comfrey—speed post-surgical healing, decrease pain and post-operative cramping, and increase erythropoiesis

And optionally a combination of some or all of the following:
Chinese angelica—to decrease indigestion and heartburn
Clove bud—antiemetic
Meadow sweet—anti nausea
Cardamom (e.g., cardamom seed)—for indigestion and nausea and vomiting
Peppermint—anti nausea, antiemetic, flavorant
Fennel—anti-indigestion
Cinnamon—improves digestion and helps with nausea and vomiting, flavorant
Goldthread root—anti nausea
Cannabis—anti nausea
Caraway—soothes upset stomachs
Tangerine peel—forward propulsion of digestive system, flavor
Alfalfa herb—anitemetic
Anise seed and Hyssop—antiemetic, flavorant
Chamomile Flower—antiemetic (e.g., effective against vomiting and nausea)
Raspberry leaf—antiemetic, flavorant
Red clover flower—antiemetic
Spearmint herb—antiemetic, flavorant
Coqueliquot or wild corn poppy—antiemetic, colorant, flavorant
Slippery elm—for indigestion
Ginger—anti motion sickness anti post-operative nausea and vomiting
Hops—antacid

And optionally a combination of some or all of the following more antianxiety for stressful occasions, sedation for preoperative preparation:
Chamomile flower—antianxiety, mild sedative
Lemon balm herb—sedative, flavorant
Lemongrass leaf—sedative, flavorant
Lemon Verbena herb—sedative, flavorant
Linden Flower—sedative
Mullein Flower—sedative
Basil leaf—sedative
Hysop Leaf—sedative
Hops—sedation, anti-SNS and anti-GERD
Passion flower—mild sedative
American Valerian Root—mild sedative, anti-stress
Valerian—sedative
Mimosa flowers—sedative
Bitter orange peel, leaf or flowers—sedative, flavorant
Jasmin13 sedative, somnifacient
Lavender—relaxant or sedative
Lotus corniculatus—sedative
Calendula—calmative
Dandelion Root—hypnotic, anti-SNS, sedative
Catnip Leaf—sedative
California Poppy—sedative More detailed effects of these active ingredients, together with suitable dosages thereof, are described in more detail below. In general, unless specifically provided, these herbal ingredients can be used at a proper dose based on their traditional usage in herbal medicines, including dose, preparation, administration frequency, preferred or prohibited combinations thereof, etc., as is known to one of skill in the relevant art.

The subject beverage can be ingested the night or the day of surgery up to 2 or 3 hrs. (e.g., up to 1 or 2 hours, up to 30-60 min., up to 15-30 min.) pre-operatively, or before any demanding procedure or human event (i.e., important speech, major business presentation, sporting event, etc.). The aim is to develop a safe beverage that will make patients and nonsurgical patients feel better before, during and after their stressful process, while simultaneously reducing their risk of Post-operative nausea and vomiting (PONV) and aspiration.

There are a large number of herbal ingredients, which permit the mixing of flavors while maintaining the properties of the beverage. The known effects of such herbal ingredients, as used in the subject beverages, are described below.

### Echinacea

Echinacea is a genus of herbaceous flowering plants in the daisy family, Asteraceae. The nine species it contains are commonly called coneflowers. Echinacea is popularly believed to be an immuno-stimulator, stimulating the body's non-specific immune system and warding off infections. It has also been used as a laxative. Some species of Echinacea are used in herbal medicines, which may all be used in the instant invention.

Marketed and studied medicinal products based on Echinacea contain different species (E. purpurea, E. angustifolia, E. pallida), different organs (roots and herbs) and different preparations (extracts and expressed juice), with different chemical compositions. Echinacea is believed to have beneficial effects on host immune system, in that it reduces the chances of catching a cold by more than half, and shorten the duration of a cold by an average of 1.4 days, based on a recent study combining findings from 14 previously reported trials (Shah et al., “Evaluation of Echinacea for the prevention and treatment of the common cold: a meta-analysis,” The Lancet Infectious Diseases 7(7): 473-480, 2007). The European Medicines Agency (EMEA), after assessing the body of evidence, approved the use of expressed juice and dried expressed juice from fresh flowering aerial parts of Echinacea purpurea for the short-term prevention and treatment of the common cold.

In certain embodiments, Echinacea used in the instant invention comprises one, two, or all three of its immune-enhancing chemicals: polysaccharides, alkylamides and cichoric acid.

In certain embodiments, the dosage of Echinacea used in the instant invention, measured as equivalent of dried Echinacea angustifolia root, is at least about 3, 4, 5, or 10 grams per day per adult of average weight (e.g., about 60-75 kg).

### Ginseng

Ginseng includes any one of 11 species of slow-growing perennial plants with fleshy roots, belonging to the genus Panax of the family Araliaceae. These ginsengs, or true ginsengs, are characterized by the presence of ginsenosides.

Siberian ginseng (Eleutherococcus senticosus) is in the same family, but not genus, as true ginseng. Like ginseng, it is considered to be an adaptogenic herb. The active compounds in Siberian ginseng are eleutherosides, not ginsenosides. Instead of a fleshy root, Siberian ginseng has a woody root.

Folk medicine attributes various benefits to oral use of American ginseng and Asian ginseng (P. ginseng) roots, including roles as an aphrodisiac, stimulant, type II diabetes treatment, or cure for sexual dysfunction in men. Ginseng root is most often available in dried form, either whole or sliced. Ginseng leaf (most often available in dried form), although not as highly prized, is sometimes also used.

Ginsenosides, unique compounds of the Panax species, are under basic and clinical research to investigate their potential for use in medicine. Meanwhile, a study showed taking Siberian Ginseng daily can increase the number of white blood cells, including activity of the T cells as well as the cytotoxic T cells and natural killer cells that eliminate invading cells and those that have been virally infected. A double blind-study with 93 volunteers with herpes simplex virus 2 (which can lead to genital herpes) found that there was a reduction (of 50%) in the number of outbreaks. Those that did occur were less severe and did not last as long. Ginseng is also known to contain phytoestrogens. Gingseng has been shown to decrease blood alcohol levels.

Ginseng may be included in small doses in the subject beverages. The common adaptogen ginsengs (P. ginseng and P. quinquefolia) are generally considered to be relatively safe even in large amounts. One of the most common and characteristic symptoms of acute overdose of Panax ginseng is bleeding. Symptoms of mild overdose may include dry mouth and lips, excitation, fidgeting, irritability, tremor, palpitations, blurred vision, headache, insomnia, increased body temperature, increased blood pressure, edema, decreased appetite, dizziness, itching, eczema, early morning diarrhea, bleeding, and fatigue. Thus the dose of the ginseng in the subject beverage preferably avoids most, if not all of the adverse side effects in a majority of the patients.

### Comfrey

Comfrey (also comphrey) is used as a fertilizer and as an herbal medicine. The most commonly used species is Russian comfrey Symphytum×uplandicum, which is a cross or hybrid of Symphytum officinale (Common Comfrey) and Symphytum asperum (Rough Comfrey).

Comfrey enjoys a long and widespread usage as a therapeutic herb. One of the most common modern-day uses of Comfrey extract is as a skin treatment. The plant contains the small organic molecule allantoin, which is thought to stimulate cell growth and repair while simultaneously depressing inflammation. The allantoin contained in the plant is thought to help replace and thus repair cells in the body through its proliferant properties. Comfrey was used in an attempt to treat a wide variety of ailments ranging from bronchial problems, broken bones, sprains, arthritis, gastric and varicose ulcers, severe burns, acne and other skin conditions. It was reputed to have bone and teeth building properties in children, and have value in treating “many female disorders”. Constituents of comfrey also include mucilage, steroidal saponins, tannins, pyrrolizidine alkaloids, inulin, and proteins. Most recently, in a placebo-controlled study comfrey was found to decrease back pain when used topically.

Angelica sinensis (Chinese angelica)

The dried root of Angelica sinensis is commonly known as Chinese angelica, also commonly known as “dong quai” or “female ginseng,” is a herb from the family Apiaceae, indigenous to China. It is widely used in Chinese traditional medicine for gynecological ailments, fatigue, mild anemia, and high blood pressure. The plant's phytochemicals consist of coumarins, phytosterols, polysaccharides, ferulate, and flavonoids. It also has antioxidant activity. It can be used in the instant invention to decrease indigestion and heartburn.

The dosage to be used should be consistent with the dose used in Chinese medicine or less (e.g., 80%, 70%, 60%, 50%, 40%, 30%, 20% or 10%). For example, angelica root can be given at doses of 3 to 6 g/day of the crude root.

### Clove Bud

Cloves are the aromatic flower buds of a tree in the family Myrtaceae, Syzygium aromaticum. Cloves are used in the cuisine of various cultures (e.g., Asian, African, and the Near and Middle East), lending flavor to food items such as meats, curries, and marinades. They may also serve as complement to fruits (e.g., apples, pears, or rhubarb). Thus cloves may be used as a flavorant in the instant invention.

Cloves have been used in traditional medicines, such as Indian Ayurvedic medicine, Chinese medicine, and western herbalism and dentistry where the essential oil is used as an anodyne (painkiller) for dental emergencies. Cloves are also used as a carminative, to increase hydrochloric acid in the stomach and to improve peristalsis. Cloves are also said to be a natural anthelmintic. The essential oil is used in aromatherapy when stimulation and warming are needed, especially for digestive problems. Topical application over the stomach or abdomen are said to warm the digestive tract.

In Chinese medicine, cloves or ding xiang are considered acrid, warm, and aromatic, entering the kidney, spleen and stomach meridians, and are notable in their ability to warm the middle, direct stomach qi downward, to treat hiccough and to fortify the kidney yang. Because the herb is so warming, it is contraindicated in any persons with “fire” symptoms and according to classical sources should not be used for anything except cold from yang deficiency. As such, it is used in formulas for impotence or clear vaginal discharge from yang deficiency, for morning sickness together with ginseng and patchouli, or for vomiting and diarrhea due to spleen and stomach coldness. The clove buds can be used in the instant invention for its antiemetic effect. They have also been found to have antioxidant properties.

Eugenol, one of the chemicals contained in clove oil, has been used historically in the West for dental pain and has been approved for human use by the U.S. Food and Drug Administration (FDA). Clove oil seems safe when applied to the skin. In addition, clove oil has been used in preparation of some toothpastes, laxative pills, and Clovacaine solution, which is a local anesthetic used in oral ulceration and inflammation. Eugenol (or clove oil generally) is mixed with zinc oxide to form a temporary tooth cavity filling.

Eugenol comprises 72-90% of the essential oil extracted from cloves, and is the compound most responsible for the cloves' aroma. Other important essential oil constituents of clove oil include acetyl eugenol, beta-caryophyllene and vanillin, crategolic acid, tannins such as bicornin, gallotannic acid, methyl salicylate (painkiller), the flavonoids eugenin, kaempferol, rhamnetin, and eugenitin, triterpenoids such as oleanolic acid, stigmasterol, and campesterol, and several sesquiterpenes. Eugenol can be toxic in relatively small quantities - as low as 5 ml. Thus, in certain embodiments, the total amount of clove buds in the subject beverage does not exceed 5 ml equivalent of Eugenol, preferably <4 ml, 3 ml, 2 ml, or 1 ml equivalent of Eugenol.

Filipendula ulmaria (Meadowsweet)

Filipendula ulmaria, commonly known as meadowsweet or mead wort, is a perennial herb in the family Rosaceae that grows in damp meadows.

The whole herb possesses a pleasant taste and flavor, the green parts having a similar aromatic character to the flowers, leading to the use of the plant as a strewing herb, strewn on floors to give the rooms a pleasant aroma, and its use to flavor wine, beer, and many vinegars. The flowers can be added to stewed fruit and jams, giving them a subtle almond flavor.

In addition to add flavor, it has many medicinal properties. The whole plant is a traditional remedy for an acidic stomach, and the fresh root is often used in infinitesimal quantities in homeopathic preparations. Dried flowers are used in potpourri. Filipendula ulmaria flowers or herb have been used in the traditional Austrian medicine internally as tea for treatment of rheumatism, gout, infections, and fever. It can also be used as an anti-nausea agent in the beverages of the invention.

Its chemical constituents include salicylic acid, flavone glycosides, essential oils, and tannins. In 1897, Felix Hoffmann created a synthetically altered version of salicin, derived from the species, which caused less digestive upset than pure salicylic acid. The new drug, formally acetylsalicylic acid, was named aspirin by Hoffman's employer Bayer AG after the old botanical name for meadowsweet, Spiraea ulmaria. This gave rise to the class of drugs known as non-steroidal anti-inflammatory drugs (NSAIDs).

Doses of 2.5 to 3.5 g/day of flower and 4 to 5 g of herb are considered conventional. A tea may be prepared from 4 to 6 g of the dried herb and taken 3 times daily.

### Cardamom (e.g., Cardamom Seed)

Cardamom (or cardamon) refers to several plants of the similar genera Elettaria and Amomum in the ginger family Zingiberaceae.

Green cardamom is broadly used in South Asia to treat infections in teeth and gums, to prevent and treat throat troubles, congestion of the lungs and pulmonary tuberculosis, inflammation of eyelids and also digestive disorders. It also is used to break up kidney stones and gall stones, and was reportedly used as an antidote for both snake and scorpion venom. Amomum is used as a spice and as an ingredient in traditional medicine in systems of the traditional Chinese medicine. Among other species, varieties and cultivars, Amomum villosum is used in traditional Chinese medicine to treat stomach issues, constipation, dysentery, and other digestion problems. Tsaoko cardamom Amomum tsao-ko is cultivated in Yunnan, China and northwest Vietnam, both for medicinal purposes and as a spice. It can be used for alleviating indigestion, nausea and vomiting in the instant invention.

The main constituent of cardamom is the essential oil in the seeds, which oil may include α-terpineol 45%, myrcene 27%, limonene 8%, menthone 6%, β-phellandrene 3%, 1,8-cineol 2%, sabinene 2% and heptane 2% (Phytochemistry, 26, 207, 1987). Other sources report 1,8-cineol (20 to 50%), α-terpenyl acetate (30%), sabinene, limonene (2 to 14%) and borneol. In the seeds of round cardamom from Jawa (A. kepulaga), the content of essential oil is lower (2 to 4%), and the oil contains mainly 1,8 cineol (up to 70%) plus β-pinene (16%); furthermore, α-pinene, α-terpineol and humulene were found.

### Ginger (e.g., Ginger Root Extract)

Ginger or ginger root is the rhizome of the plant Zingiber officinale, also in the family Zingiberaceae. Ginger was found to be more effective than placebo for treating nausea caused by seasickness, morning sickness and chemotherapy. Some preliminary studies also showed that ginger may affect arthritis pain or have blood thinning and cholesterol lowering properties.

The characteristic odor and flavor of ginger is caused by a mixture of zingerone, shogaols and gingerols, volatile oils that compose one to three percent of the weight of fresh ginger. In laboratory animals, the gingerols increase the motility of the gastrointestinal tract and have analgesic, sedative, antipyretic and antibacterial properties. Ginger contains up to three percent of a fragrant essential oil whose main constituents are sesquiterpenoids, with (−)-zingiberene as the main component. Smaller amounts of other sesquiterpenoids (β-sesquiphellandrene, bisabolene and farnesene) and a small monoterpenoid fraction (β-phellandrene, cineol, and citral) have also been identified.

The traditional medical form of ginger historically was called Jamaica ginger; it was classified as a stimulant and carminative and used frequently for dyspepsia, gastroparesis, slow motility symptoms, constipation, and colic. It was also frequently employed to disguise the taste of medicines. Some studies indicate ginger may provide short-term relief of pregnancy-related nausea and vomiting. Tea brewed from ginger is a common folk remedy for colds. Ginger ale and ginger beer are also drunk as stomach settlers in countries where the beverages are made.

Ginger is recognized as safe by the FDA (i.e., on the FDA's “generally recognized as safe” list), and is sold as an unregulated dietary supplement in the U.S.

Similar species that can also be used in the beverages of the invention include Myoga (Zingiber mioga Roscoe); galangal (also called Thai ginger); galangal, fingerroot (Boesenbergia rotunda), Chinese ginger, or the Thai krachai; Asarum canadense (also known as “wild ginger”).

### Peppermint

Peppermint (Mentha×piperita, also known as M. balsamea Willd.) is a hybrid mint, a cross between watermint and spearmint. Peppermint has a long tradition of medicinal use, dating as far back as ten thousand years ago. Peppermint has a high menthol content, and is often used in tea and for flavouring ice cream, confectionery, chewing gum, and toothpaste. The oil also contains menthone and menthyl esters, particularly menthyl acetate. Dried peppermint typically has 0.3-0.4% of volatile oil containing menthol (7-48%), menthone (20-46%), menthyl acetate (3-10%), menthofuran (1-17%) and 1,8-cineol (3-6%). Peppermint oil also contains small amounts of many additional compounds including limonene, pulegone, caryophyllene and pinene. The chemical composition of the essential oil from peppermint (Mentha×piperita L.) was found to contain menthol (40.7%), menthone (23.4%), and (+/−)-menthyl acetate, 1,8-cineole, limonene, beta-pinene and beta-caryophyllene.

Menthol activates cold-sensitive TRPM8 receptors in the skin and mucosal tissues, and is the primary source of the cooling sensation that follows the topical application of peppermint oil. Peppermint is commonly used to soothe or treat symptoms such as nausea, vomiting, abdominal pain, indigestion, irritable bowel, and bloating. It is also used in aroma therapy. One animal study has suggested that Peppermint may have radioprotective effects in patients undergoing cancer treatment. The aroma of peppermint has been found to enhance memory and alertness.

In 2007, Italian investigators reported that 75% of the patients in their study who took peppermint oil capsules for four weeks had a major reduction in irritable bowel syndrome (IBS) symptoms, compared with just 38% of those who took a placebo (Cappello et al., “Peppermint oil (Mint Oil) in the treatment of irritable bowel syndrome: A prospective double blind placebo-controlled randomized trial”. Digestive and Liver Disease 39(6): 530-6, 2007). A second study conducted in Iran in 2010 found similar results.

According to the German Commission E monographs, peppermint oil (as well as peppermint leaf) has been used internally as an antispasmodic (upper gastrointestinal tract and bile ducts) and to treat irritable bowel syndrome, catarrh of the respiratory tract, and inflammation of the oral mucosa. Externally, peppermint oil has been used for myalgia and neuralgia. According to the German Commission E, peppermint oil may also act as a carminative, cholagogue, antibacterial, and secretolytic, and it has a cooling action.

Enteric-coated peppermint oil capsules (Colpermin) been used as an orally administered antispasmodic premedication in colonoscopy. The capsules were found beneficial in reducing total procedure time, reducing colonic spasm, increasing endoscopist satisfaction and decreasing pain in patients during colonoscopy. Similarly, some earlier trials also found that peppermint oil has the ability to reduce colicky abdominal pain due to IBS with an NNT (number needed to treat) around 3.1, but the oil is an irritant to the stomach in the quantity required and therefore needs wrapping for delayed release in the intestine. This could also be achieved by using the whole herb or leaves rather than the volatile components alone.

Peppermint oil is also an ingredient in a “natural” haemorrhoid treatment.

### Fennel

Fennel (Foeniculum vulgare) is a plant species in the genus Foeniculum, in the family Apiaceae. It is a highly aromatic and flavorful herb with culinary and medicinal uses, and is one of the primary ingredients of absinthe.

Fennel contains anethole, which itself or its polymers act as phytoestrogens. The essence of fennel can be used as a safe and effective herbal drug for primary dysmenorrhea. Fennel is widely employed as a carminative, both in humans and in veterinary medicine (e.g., dogs), to treat flatulence by encouraging the expulsion of intestinal gas. Anethole is responsible for the carminative action.

On account of its aromatic and carminative properties, fennel fruit is chiefly used medicinally with purgatives to allay their tendency to griping, and, for this purpose, forms one of the ingredients of the well-known compound Liquorice Powder. Fennel water has properties similar to those of anise and dill water: mixed with sodium bicarbonate and syrup, these waters constitute the domestic ‘gripe water’, used to correct the flatulence of infants. Volatile oil of Fennel has these properties in concentration. Fennel tea, formerly also employed as a carminative, is made by pouring half a pint of boiling water on a teaspoonful of bruised fennel seeds. Fennel can also be made into a syrup to treat babies with colic (formerly thought to be due to digestive upset), but long-term ingestion of fennel preparations by babies is a known cause of thelarche.

Fennel may also be an effective diuretic and a potential drug for treatment of hypertension.

### Cinnamon

Cinnamon is a spice obtained from the inner bark of several trees from the genus Cinnamomum that is used in both sweet and savory foods.

Cinnamon has a wide range of uses, including spice (bark) or flavouring material in cooking, dessert, chocolate, and many other food items and drinks. Cinnamon has also been proposed for use as an insect repellent. Cinnamon leaf oil has been found to be very effective in killing mosquito larvae. Of the compounds found in the essential oil from cinnamon leaves, cinnamyl acetate, eugenol, and anethole, and in particular cinnamaldehyde, were found to have the highest effectiveness against mosquito larvae. Cinnamon, as a warm and dry substance, was believed by doctors in ancient times to cure snakebites, freckles, the common cold, and kidney troubles, among other ailments. Cinnamon along with garlic is also used as a fish and meat preservative; they can also be used to preserve fried and deep fried foods.

Cinnamon is used in traditional medicine, and several studies have tested chemicals extracted from cinnamon for various possible medicinal effects. An extract of Cinnamomum cassia had an effect on HIV-1. Another study found that eugenol, a chemical found in cinnamon essential oils, and in other plants, inhibited the replication of the virus causing herpes in vitro. The compound cinnzeylanine, from C. zeylanicum, also had antiviral properties in a model system using silkworm cells.

Two studies have shown that including cinnamon and cinnamon extract in the diet may help type 2 diabetics to control blood glucose levels. Apart from the most common flavanol (epi)catechin and (epi)afzelechin, cinnamon proanthocyanidins contain (epi)catechin gallate and (epi)gallocatechin units. Furthermore, these proanthocyanidins are bioavailable and may have an effect on the target tissues. Pharmacological experiments suggest that dietary cinnamon-derived cinnamic aldehyde (cinnamaldehyde) activates the Nrf2-dependent antioxidant response in human epithelial colon cells and may therefore represent an experimental chemopreventive dietary factor targeting colorectal carcinogenesis. Recent research documents anti-melanoma activity of cinnamic aldehyde observed in cell culture and a mouse model of human melanoma. A 2011 study isolated a substance (CEppt) in the cinnamon plant that inhibits development of Alzheimer's disease in mice. CEppt, an extract of cinnamon bark, seems to treat a mouse model of Alzheimer's disease. Cinnamon can also be used in the instant beverage to improve digestion and to help with nausea and vomiting.

Ground cinnamon is generally given at dosages of 1 to 1.5 g/day in studies of diabetes without reported adverse reactions.

### Coptis (Goldthread or Canker Root)

Coptis is a genus of between 10-15 species of flowering plants in the family Ranunculaceae. Coptis teeta is used as a medicinal herb in China and the Himalayan regions of India where it is used as a bitter tonic for dyspepsia. It is also believed to help insomnia in Chinese herbology. It can be used as an anti-nausea agent in the instant beverage. The roots of this plant contain the bitter alkaloid berberine. The dried roots (goldthread) were commercially marketed in Canada until the 1950s or early 60s.

### Cannabis

Cannabis is a genus of flowering plants that includes three putative varieties, Cannabis sativa, Cannabis indica, and Cannabis ruderalis. Cannabis has long been used for medicinal purposes, and as a recreational drug. Marijuana consists of the dried flowers of Cannabis plants selectively bred to produce high levels of THC and other psychoactive cannabinoids. Various extracts including hashish and hash oil are also produced from the plant.

Cannabis is a popular recreational drug around the world. The psychoactive effects of Cannabis are known to have a biphasic nature. Primary psychoactive effects include a state of relaxation, and to a lesser degree, euphoria from its main psychoactive compound, tetrahydrocannabinol. Secondary psychoactive effects, such as a facility for philosophical thinking, introspection and metacognition have been reported amongst cases of anxiety and paranoia. Finally, the tertiary psychoactive effects of the drug cannabis, can include an increase in heart rate and hunger, believed to be caused by 11-OH-THC, a psychoactive metabolite of THC produced in the liver.

Medical cannabis (or medical marijuana) refers to the use of cannabis and its constituent cannabinoids, such as tetrahydrocannabinol (THC), as medical therapy to treat disease or alleviate symptoms. Cannabis has been used to reduce nausea and vomiting in chemotherapy and people with AIDS, and to treat pain and muscle spasticity.

### Caraway

Caraway (Carum carvi), also known as meridian fennel or Persian cumin, “Shahi Jeera,” is a biennial plant in the family Apiaceae. The fruits, usually used whole, have a pungent, anise-like flavor and aroma that comes from essential oils, mostly carvone and limonene.

They are used as a spice in breads, especially rye bread. Caraway is also used in desserts, liquors, casseroles, curry, Indian dishes and other foods.

Caraway also has a long tradition of medical uses, primarily for stomach complaints. Emerging and ongoing research from Arabic regional studies suggest Carum carni use as an endocrine function support agent, specifically related to thyroid disorders and auto immune disease (see Hashimoto's thyroiditis). Thus caraway may be used to soothe upset stomachs in the instant beverage.

Caraway is “generally recognized as safe” by the US Food and Drug Administration. It is usually well tolerated. Doses of 20-100 mg may be used.

### Tangerine Peel

The tangerine (Citrus tangerina) is an orange-colored citrus fruit closely related to the mandarin orange (Citrus reticulata). The peel of the latter (Citrus reticulata) is also called Chen pi in Chinese.

The use of the tangerine peel in ancient times was mainly for the cure of cough, colds and many other respiratory illnesses. The tangerine peel is used fresh as tea or as a refreshing drink to relieve stress and increase energy and vigor. The peel is also used as a poultice for wounds and burns.

In modern times, the tangerine peel is used to dry up mucus in the upper and lower respiratory system. It is effective in liquefying secretions for expulsion as well. For the gastrointestinal system, the tangerine peel is dried and ground to aid digestion, to treat diarrhea, stomach pain, nausea and also episodes of dyspepsia. The tangerine peel is very effective in the treatment of digestive problems because it naturally increases the secretion of the gastric juices and is also helpful in relaxing the smooth muscles of the gastrointestinal tract. Chinese medicine also believes that the tangerine peel moves the body's qi downward therefore it relieves hiccups and vomiting as well. Thus it can be used in the instant beverage to promote forward propulsion of the digestive system.

There are several studies that support the high bioflavonoid content of the tangerine peel as being beneficial in the strengthening of the epithelial cells making them resistant to cancer cells. This could be a factor in determining the effect of tangerine peel supplements in the prevention of cancer. The tangerine peel is also beneficial in weight loss. It also lowers blood cholesterol, blood pressure and thus is indirectly responsible for weight loss.

In certain embodiments, tangerine essential oil may be used in place of tangerine peel.

### Alfalfa Herb

Alfalfa has been used as an herbal medicine for over 1,500 years. In early Chinese medicines, physicians used young alfalfa leaves to treat disorders related to the digestive tract and the kidneys. In Ayurvedic medicine, physicians used the leaves for treating poor digestion. Alfalfa may be used for its antiemetic effect in the instant invention.

### Anise Seed

Anise (Pimpinella anisum), also called aniseed, is a flowering plant in the family Apiaceae native to the eastern Mediterranean region and Southwest Asia. Typical values for the main constituents of anise: Moisture: 9-13%; Protein: 18%; Fatty oil: 8-23%; Essential oil: 2-7%; Starch: 5%; N-free extract: 22-28%; and Crude fibre: 12-25%. Essential oil yielded by distillation is generally around 2-3%.

Like fennel, Anise contains anethole, a phytoestrogen. Anise has also been used to treat menstrual cramps. The main use of anise in European herbal medicine was for its carminative effect. In the instant invention, anise may be used for its antiemetic effect as well as a flavorant.

Hyssop (Anise Hyssop or Agastache foeniculum (Also Called Blue Giant Hyssop))

Agastache foeniculum (blue giant hyssop), commonly called anise hyssop, blue giant hyssop, Fragrant giant hyssop, or the lavender giant hyssop, is a species of perennial plant in the mint family (Lamiaceae).

Anise hyssop was used medicinally by Native Americans for cough, fevers, wounds, diarrhea. Its soft, anise-scented leaves are used as a seasoning, and can be crumbled in salad. Anise hyssop may be used for its antiemetic effect, anti-SNS effect, as well as a flavorant in the instant invention.

Hyssop leave is also used for its sedative effect in the instant invention.

### Raspberry Leaf

The raspberry is the edible fruit of a multitude of plant species in the genus Rubus of the rose family, most of which are in the subgenus Idaeobatus. Raspberry leaves can be used fresh or dried in tisanes. They have an astringent flavor, and in herbal medicine are reputed to be effective in regulating menses.

Raspberry leaf may be used for its antiemetic effect, as well as a flavorant in the instant invention.

Trifolium pratense (Red Clover)

Trifolium pratense (red clover) is a species of clover, native to Europe, Western Asia and northwest Africa. Red clover may be used for its antiemetic or anti-nausea/vomiting effect in the instant invention.

Mentha spicata (Spearmint or Spear Mint)

Spearmint is a species of mint native to much of Europe and southwest Asia. Spearmint tea may be used as a treatment for hirsutism in women. Its anti-androgenic properties reduce the level of free testosterone in the blood, while leaving total testosterone and DHEA unaffected. It can also be used to treat a variety of digestive ailments, including stomachache and gas. Spearmint may be used for its antiemetic effect, as well as a flavorant in the instant invention.

### Coquelicot or Wild Corn Poppy

Coquelicot is a shade of red, and was originally a French vernacular name for the wild corn poppy, Papaver rhoeas, which is a species of flowering plant in the poppy family, Papaveraceae. Coquelicot may be used for its antiemetic effect, as well as a colorant or a flavorant in the instant invention.

Ulmus rubra (Slippery Elm)

Ulmus rubra is a species of elm native to eastern North America. It has various traditional medicinal uses. The mucilaginous inner bark of the slippery elm has long been used as a demulcent, and is still produced commercially for this purpose in the United States with approval for sale as a nutritional supplement by the U.S. Food and Drug Administration. Sometimes slippery elm leaves are dried and ground into a powder, then made into a tea. Both slippery elm gruel and tea may help to soothe the digestive tract. Tea made of inner bark of slippery elm is considered to be beneficial for psoriasis because of its demulcent effect on intestines. Slippery elm may be used in the instant beverage to prevent or alleviate indigestion.

Slippery elm inner bark has been used for treatment of ulcers at doses of 1.5 to 3 g/day. It is commonly decocted with ethyl alcohol.

Chamomile (Matricaria recutita)

Chamomile or camomile is the common name for several daisy-like plants of the family Asteraceae that are commonly used to make a herb tea that can help to induce sleep.

Major chemical compounds present within chamomile include apigenin and alpha-bisabolol. Other classes of chemical compounds found within the chamomile plant include: sesquiterpenes, terpenoids, flavonoids, coumarins such as herniarin and umbelliferone, phenylpropanoids such as chlorogenic acid and caffeic acid, flavones such as apigenin and luteolin, flavanols such as quercetin and rutin, and polyacetylenes.

Apigenin has demonstrated strong chemopreventive effects, while alpha-bisabolol has been shown to have antiseptic properties, anti-inflammatory properties, and has also been demonstrated to reduce pepsin secretion without altering secretion of stomach acid. Chemical compounds present within chamomile have demonstrated the ability to bind GABA receptors, modulate monoamine neurotransmission, and have displayed neuroendocrine effects. Umbelliferone has been shown to be fungistatic. Coumarin compounds present in chamomile such as herniarin and umbelliferone may have blood-thinning properties, and there is some evidence that chamomile may interact with other medications causing drug-drug interactions.

Chamomile has been used for inflammation associated with hemorrhoids when applied topically. Evidence also supports the claim that chamomile possesses anxiolytic (anti-anxiety) properties and may have clinical applications in the treatment of stress and insomnia. Chemical components of chamomile extract have demonstrated anti-inflammatory, antihyperglycemic, antigenotoxic, and anticancer properties when examined in vitro and in animal studies.

Chamomile can be used as an antianxiety, and/or a mild sedative ingredient in the instant beverage.

Chamomile flower is also antiemetic (e.g., effective against vomiting and nausea), and may also be used as anti-SNS ingredient in the instant invention.

### Lemon Balm

Lemon balm (Melissa officinalis), also known as balm or balm mint, is a perennial herb in the mint family Lamiaceae.

In the traditional Austrian medicine Melissa officinalis leaves have been prescribed for internal (as tea) or external (essential oil) application for the treatment of disorders of the gastrointestinal tract, nervous system, liver and bile. Lemon balm is the main ingredient of Carmelite Water, which is still for sale in German pharmacies. Lemon balm essential oil is very popular in aromatherapy. The essential oil is commonly co-distilled with lemon oil, citronella oil, or other oils.

High doses of purified lemon balm extracts were found to be effective in the amelioration of laboratory-induced stress in human subjects, producing “significantly increased self-ratings of calmness and reduced self-ratings of alertness.” The authors further report a “significant increase in the speed of mathematical processing, with no reduction in accuracy” following the administration of a 300 mg dose of extract. Thus in certain embodiment, the dose of lemon balm in the instant beverage is about 300 mg, as a sedative ingredient.

Lemon balm is believed to inhibit the absorption of the thyroid medication thyroxine. Recent research found a daily dose of the tea reduced oxidative stress status in radiology staff that were exposed to persistent low-dose radiation during work. After only 30 days of taking the tea daily, researchers found Lemon balm tea resulted in a significant improvement in plasma levels of catalase, superoxide dismutase, and glutathione peroxidase and a marked reduction in plasma DNA damage, myeloperoxidase, and lipid peroxidation.

Lemon balm is also used medicinally as an herbal tea, or in extract form. It is used as an anxiolytic, mild sedative, or calming agent. At least one study has found it to be effective at reducing stress. Lemon balm extract was identified as a potent in vitro inhibitor of GABA transaminase, which explains anxiolytic effects. The major compound responsible for GABA transaminase inhibition activity in lemon balm was then found to be rosmarinic acid.

Lemon balm and preparations thereof also have been shown to improve mood and mental performance. These effects are believed to involve muscarinic and nicotinic acetylcholine receptors. Positive results have been achieved in a small clinical trial involving Alzheimer patients with mild to moderate symptoms. Essential oils obtained from Melissa officinalis leaf showed high acetylcholinesterase and butyrylcholinesterase co-inhibitory activities.

Its antibacterial properties have also been demonstrated scientifically. The extract of lemon balm was also found to have exceptionally high antioxidant activity. Lemon balm is mentioned in the scientific journal Endocrinology, where it is explained that Melissa officinalis exhibits anti-thyrotropic activity, inhibiting TSH from attaching to TSH receptors, hence making it of possible use in the treatment of Graves' disease or hyperthyroidism.

Lemon balm contains eugenol, tannins, and terpenes. Melissa officinalis also contains 1-octen-3-ol, 10-alpha-cadinol, 3-octanol, 3-octanone, alpha-cubebene, alpha-humulene, beta-bourbonene, caffeic acid, caryophyllene, caryophyllene oxide, catechinene, chlorogenic acid, cis-3-hexenol, cis-ocimene, citral A, citral B, citronellal, copaene, delta-cadinene, eugenyl acetate, gamma-cadinene, geranial, geraniol, geranyl acetate, germacrene D, isogeranial, linalool, luteolin-7-glucoside, methylheptenone, neral, nerol, octyl benzoate, oleanolic acid, pomolic acid, protocatechuic acid, rhamnazine, rosmarinic acid, rosmarinin acid, stachyose, succinic acid, thymol, trans-ocimene, ursolic acid, and harmine.

Crude lemon balm herb is typically dosed at 1.5 to 4.5 g/day. Doses of 600 to 1,600 mg extract have been studied in trials. A standardized preparation of lemon balm (80 mg) and valerian extract (160 mg) has been given 2 or 3 times/day as a sleep aid, and has also been studied in children. A 1% extract cream has been studied as a topical agent for treatment of herpes virus lesions.

### Lemongrass (Cymbopogon) Leaf

Lemongrass is native to India and tropical Asia. It is widely used as a herb in Asian cuisine, and has a subtle citrus flavor. Lemongrass oil is used as a pesticide and a preservative. Research shows that lemongrass oil has antifungal properties. Lemon grass is also used as an addition to tea, and in preparations such as kadha, which is a traditional herbal soup used against coughs, colds, etc. It has medicinal properties and is used extensively in Ayurvedic medicine. It is supposed to help with relieving cough and nasal congestion.

Lemongrass may be used as sedative and/or flavorant in the instant invention.

### Lemon Verbena Herb

Aloysia citrodora, commonly known as lemon verbena and lemon beebrush, is a species of flowering plant in the verbena family Verbenaceae, native to western South America.

Lemon verbena leaves are used to add a lemon flavor to food items. It also is used to make herbal teas, or added to standard tea in place of actual lemon. In addition, it has anti-Candida albicans activity. Moderate antioxidant supplementation with lemon verbena extract protects neutrophils against oxidative damage, decreasing the signs of muscular damage in chronic running exercise without blocking the cellular adaptation to exercise. Lippia citriodora extract shows antioxidant properties that could play an important role in modulating GSH-reductase activity in lymphocytes and erythrocytes and protecting plasma from exercise oxidative damage.

Lemon verbena extract containing 25% verbascoside showed strong antioxidant capacity, especially in a lipophilic environment, which was higher than expected as concluded from the antioxidant capacity of pure verbascoside, probably due to synergistic effects.

Lemon verbena may be used as a sedative and/or flavorant in the instant invention.

### Linden Flower

Linden is an herb native to North America, and parts of Asia and Europe, and has traditionally been used as an herbal remedy in Europe for centuries. Linden flowers contain a few different health benefits, but are most commonly used for treating cold or cough, and related symptoms or ailments. Other health benefits of linden flowers include reducing stress on the body, as well as having a light calming effect, serving to help reduce anxiety. Various studies have also shown the linden flower to be successful at treating stomach pains and relieving gas pressure. The herbal remedy linden flowers are commercially available.

Linden flower may be used for its sedative and/or anti-SNS effect in the instant invention.

Mullein Flower

Verbascum thapsus (great mullein or common mullein) is a species of mullein native to Europe, northern Africa and Asia, and introduced in the Americas and Australia.

It is widely used for herbal remedies with emollient and astringent properties. It is known to possess anti-inflammatory, anti-tumor, antiviral, antifungal, antibacterial, expectorant, and analgesic properties. It is especially recommended for coughs and related problems, but also used in topical applications against a variety of skin problems.

Mullein flower may be used for its sedative effect in the instant invention.

### Basil Leaf

Basil, Thai basil, or sweet basil, is a common name for the culinary herb Ocimum basilicum of the family Lamiaceae (mints), sometimes known as Saint Joseph's Wort in some English-speaking countries.

Scientific studies in vitro have established that compounds in basil oil have potent antioxidant, antiviral, and antimicrobial properties, and potential for use in treating cancer. In addition, basil has been shown to decrease the occurrence of platelet aggregation and experimental thrombus in mice. It is traditionally used for supplementary treatment of stress, asthma and diabetes in India.

Basil leaf may be used for its sedative effect in the instant invention.

### Hops

Hops are the female flowers (also called seed cones or strobiles) of the hop plant, Humulus lupulus. They are used primarily as a flavoring and stability agent in beer, to which they impart a bitter, tangy flavor, though hops are also used for various purposes in other beverages and herbal medicine.

When used in herbal medicine, hops can be used to treat anxiety, restlessness, and insomnia, and animal research has shown a sedative effect. Thus hops may be used for its sedative effects in the subject beverage. The relaxing effect of hops may be due, in part, to the specific chemical component dimethylvinyl carbinol. In addition, hops are of interest for hormone replacement therapy, and are used in preparations for relief of menstruation-related problems.

Hops contain humulone, isohumulone, and humulene, which are bitter-tasting compounds. It also contains the natural phenols xanthohumol, isoxanthohumol, and the most estrogenic phytoestrogen known, 8-prenylnaringenin.

Hops has been used as a mild sedative or sleep aid, with the dried strobile given in doses of 1.5 to 2 g. An extract combination with valerian, Ze 91019 (ReDormin, Ivel) has been studied at a hops dose of 60 mg for insomnia. Hops can also be used for its anti-SNS and anti-GERD effects.

### Passiflora (Passion Flowers)

Passiflora, known also as the passion flowers or passion vines, is a genus of about 500 species of flowering plants, the namesakes of the family Passifloraceae.

P. incarnata (maypop) leaves and roots have a long history of use among Native Americans in North America and were adapted by the European colonists. The fresh or dried leaves of maypop are used to make a tea that is used to treat insomnia, hysteria, and epilepsy, and is also valued for its analgesic properties. P. edulis (passion fruit) and a few other species are used in Central and South America for similar purposes. Once dried, the leaves can also be smoked.

Many species have been found to contain beta-carboline harmala alkaloids, which are MAO inhibitors with anti-depressant properties. The flower and fruit have only traces of these chemicals, but the leaves and the roots are often more potent and have been used to potentiate the effects of mind-altering drugs. The most common of these alkaloids is harman (1-methyl-9H-β-carboline), but harmaline (4,9-dihydro-7-methoxy-l-methyl-3H-pyrido[3,4-b]indole), harmalol (1-methyl-2,3,4,9-tetrahydropyrido[3,4-b]indol-7-one), harmine (7-methoxy-1-methyl-9H-pyrido[3,4-b]indole) and harmol were found. The species known to bear such alkaloids include: P. actinea, P. alata (winged-stem passion flower), P. alba, P. bryonioides (cupped passion flower), P. caerulea (blue passion flower), P. capsularis, P. decaisneana, P. edulis (passion fruit), P. eichleriana, P. foetida (stinking passion flower), P. incarnata (maypop), P. quadrangularis (giant granadilla), P. ruberosa, P. subpeltata and P. warmingii, each of which may be used in the subject beverage.

Other compounds found in passion flowers are coumarins (e.g., scopoletin and umbelliferone), maltol, phytosterols (e.g. lutenin) and cyanogenic glycosides (e.g. gynocardin) which render some species, i.e., P. adenopoda, somewhat poisonous. Many flavonoids and their glycosides have been found in Passiflora, including apigenin, benzoflavone, homoorientin, 7-isoorientin, isoshaftoside, isovitexin (or saponaretin), kaempferol, lucenin, luteolin, n-orientin, passiflorine (named after the genus), quercetin, rutin, saponarin, shaftoside, vicenin and vitexin. Maypop, Blue Passion Flower (P. caerulea), and perhaps others contain chrysin, a flavone with confirmed anxiolytic and anti-inflammatory, and supposed aromatase inhibitor properties. Also documented to occur at least in some Passiflora in quantity are the hydrocarbon nonacosane and the anthocyanidin pelargonidin-3-diglycoside.

As regards organic acids, the genus is rich in formic, butyric, linoleic, linolenic, malic, myristic, oleic and palmitic acids as well as phenolic compounds, and the amino acid a-alanine. Esters like ethyl butyrate, ethyl caproate, n-hexyl butyrate and n-hexyl caproate give the fruits their flavor and appetizing smell. Sugars, contained mainly in the fruit, are most significantly d-fructose, d-glucose and raffinose. Among enzymes, Passiflora was found to be rich in catalase, pectin methylesterase and phenolase.

The medical utility of very few species of Passiflora has been scientifically studied. In initial trials for treatment of generalized anxiety disorder, maypop extract performed as well as oxazepam but with fewer short-term side effects. Passionflower herb (Passiflorae herba) from P. incarnata is official in the European Pharmacopoeia. The herbal drug should contain not less than 1.5% total flavonoids expressed as vitexin. It is used in sedative tea mixtures with other calming herbs.

A daily dose of 4 to 8 g of passion flower is typical. It can be used as mild sedative and for its anti-SNS effect.

### Valerian and American Valerian Root

Valerian (Valerian officinalis, Valerianaceae) is a hardy perennial flowering plant, with heads of sweetly scented pink or white flowers that bloom in the summer months. Native to Europe and parts of Asia, valerian has been introduced into North America.

In pharmacology and herbal medicine, valerian is the name of a herb or dietary supplement prepared from roots of the plant. Crude extract of the root is often sold in the form of capsules. Valerian root has sedative and anxiolytic effects. It can also be classified as a drug, and its consumption produces a sedative or medicinal effect. These effects are suspected to be mediated through the GABA receptor.

Known compounds detected in valerian that may contribute to its method of action include:
Alkaloids: actinidine, chatinine, shyanthine, valerianine, and valerine
Isovaleramide, which may be created in the extraction process
Gamma-aminobutyric acid (GABA)
Isovaleric acid
Iridoids, including valepotriates: isovaltrate and valtrate
Sesquiterpenes (contained in the Volatile oil): valerenic acid, hydroxyvalerenic acid and acetoxyvalerenic acid.
Flavanones: hesperidin, 6-methylapigenin and linarin

Because of valerian's historical use as a sedative, anticonvulsant, migraine treatment and pain reliever, most basic science research has been directed at the interaction of valerian constituents with the GABA neurotransmitter receptor system. The mechanism of action of valerian in general, and as a mild sedative in particular has not been fully elucidated, but it is generally believed that some of the GABA-analogs particularly valerenic acids as components of the essential oil along with other semi-volatile sesquiterpenoids appear to have some affinity for the GABA_{A} receptor, a class of receptors on which benzodiazepines are known to act. Valeric acid, which is responsible for the typical odor of mostly older valerian roots, does not have any sedative properties.

Valerian also contains isovaltrate, which has been shown to be an inverse agonist for adenosine A₁ receptor sites. This action likely does not contribute to the herb's sedative effects, which would be expected from an agonist, rather than an inverse agonist, at this particular binding site. Hydrophilic extractions of the herb commonly sold over-the-counter, however, probably do not contain significant amounts of isovaltrate.

The chief constituent of valerian is a yellowish-green to brownish-yellow oil which is present in the dried root, varying from 0.5 to 2.0 percent, though an average yield rarely exceeds 0.8 percent. Valerian, when used in tea or the instant beverage, should not be prepared with boiling water, as this may drive off the lighter oils.

Valerian is most often used to treat insomnia. It can be considered an alternative treatment instead of hypnotic drugs. It is also sometimes used as an alternative for sedatives, such as benzodiazepines in the treatment of certain anxiety disorders.

In addition to being used for the treatment of insomnia and other sleep disorders, valerian has also been used to treat anxiety and gastrointestinal pain caused by irritable bowel syndrome. It has also been used in alternative medicine to treat symptoms such as nervous tension, excitability, and stress, as well as intestinal colic or cramps and as a muscle relaxant.

Oral forms are available in both standardized and unstandardized forms. Standardized products may be preferable considering the wide variation of the chemicals in the dried root, as noted above. When standardized, it is done so as a percentage of valerenic acid or valeric acid.

As to dosage, the NIH reports that doses of 400-900 mg taken from 30 minutes to two hours before bed, or a lesser dose when combined with certain other supplements, have been researched.

Because the compounds in valerian produce central nervous system depression, they should not be used with other depressants, such as alcohol, benzodiazepines, barbiturates, opiates, or antihistamine drugs. Moreover, non-pregnant adult human hepatotoxicity has been associated with short-term use (i.e., a few days to several months) of herbal preparations containing valerian and Scutellaria (commonly called skullcap). Thus in certain embodiments, the subject composition does not contain valerian or its related ingredients.

For anxiety, valeprotriates dosed at about 150 mg/day in 3 divided doses for 4 weeks has been used in a clinical trial. Other trials used the dried herb 0.5 to 2 g, extract 0.5 to 2 mL, and valerian tincture 2 to 4 mL for anxiety. For insomnia, valerian extract 400 to 600 mg/day taken 1 hour before bedtime for 2 to 4 weeks has been used in clinical trials.

### Mimosa Flowers

Mimosa (Albizia julibrissin Durazz., in the family of Fabaceae) is also commonly known as powder-puff tree or silk tree. In vitro studies document anti-cancer activity of the various julibrosides against numerous cancer cell lines. The herb is also often marketed for relieving anxiety, depression, and stress. It can be used as sedative agent in the subject beverage.

The stem bark of mimosa has been used as a sedative for hundreds of years as recorded in the Pharmacopeia of the People's Republic of China, and as an anti-inflammatory agent for swelling and pain in the lungs and to treat skin ulcers, wounds, bruises, abscesses, boils, hemorrhoids, and fractures, as well as to remove carbuncles. The dried stem bark is also used as a tonic in China and Japan. Some indigenous Korean also prepare the root as an infusion for bone diseases. In India, a chloroform and methanol seed extract has been used to treat bronchitis, asthma, leprosy, and glands infected by tuberculosis. A bark extract to treat insomnia, diuresis, asthenia, and confusion has been used in Asia. The plant's flowers have been used to treat symptoms associated with palpitations, anxiety, depression, and insomnia.

Minosa is available from commercial manufacturers, and the most common dosage forms are liquids and capsules. Manufacturers suggest 3 to 6 mL of 1:2 mimosa liquid extract daily or 1 capsule 3 times daily with meals. However, capsule formulations are proprietary herbal blends and are available in several strengths. Powders, teas, and tinctures are also available.

### Bitter Orange Peel, Leaf or Flowers

Bitter orange, Seville orange, sour orange, bigarade orange, or marmalade orange refers to a citrus tree (Citrus×aurantium) and its fruit. It is hybrid between Citrus maxima (pomelo) and Citrus reticulata (mandarin). Many varieties of bitter orange are used for their essential oil, which is used in perfume, as a flavoring and as a solvent.

Bitter orange is also employed in herbal medicine as a stimulant and appetite suppressant, due to its active ingredient, synephrine.

Bitter orange peel, leaf or flowers can be used for their sedative effect or as a flavorant in the instant invention.

### Jasmine

Jasmine (Jasminum) is a genus of shrubs and vines in the olive family (Oleaceae). Jasmine tea is consumed in China, where it is called jasmine-flower tea. Jasmine (e.g., whole jasmine flowers) can be used in the instant invention for its sedative and somnifacient effects.

### Lavender

Lavandula (common name Lavender) is a genus of flowering plants in the mint family, Lamiaceae. Commercially the plant is grown mainly for the production of essential oil of lavender. This has antiseptic and anti-inflammatory properties. These extracts are also used as fragrances for bath products.

Lavender is used extensively with herbs and aromatherapy. Infusions are believed to soothe insect bites, burns, and headaches. Bunches of lavender repel insects. In pillows, lavender seeds and flowers aid sleep and relaxation. An infusion of flowerheads added to a cup of boiling water is used to soothe and relax at bedtime. Lavender oil (or extract of Lavender) is used to treat acne when diluted 1:10 with water, rosewater, or witch hazel; it also treats skin burns and inflammatory conditions.

Lavender oil is approved for use as an anxiolytic in Germany under the name Lasea. A survey paper on lavender and the nervous system suggests that lavender oil may be an effective medicament in treatment of several neurological disorders.

Lavender may be used as a relaxant in the instant invention.

Lotus corniculatus

Lotus corniculatus (commonly known as “birdsfoot trefoil”) is a common flowering plant native to grassland temperate Eurasia and North Africa. Fresh birdsfoot trefoil contains cyanogenic glycosides, which releases small amounts of hydrogen cyanide when macerated. This is however not normally poisonous to humans, as the dose is very low, and the metabolization of cyanide is relatively quick. Condensed Tannins are also present in Lotus Corniculatus, which has been known to increase the protein absorption of the small intestine. Used in an infusion to avoid the creation of hydrogen cyanide this plant can be used as a sedative. in the instant invention.

### Calendula

Calendula or marigold, is a genus of annual and perennial herbaceous plants in the daisy family Asteraceae. Calendula species have been used traditionally as culinary and medicinal herbs. The flowers of C. officinalis contain flavonol glycosides, triterpene oligoglycosides, oleanane-type triterpene glycosides, saponins, and a sesquiterpene glucoside. The oil of C. officinalis is used as an anti-inflammatory, an anti-tumor agent, and a remedy for healing wounds. Calendula can be used as a sedative in the instant invention.

### Dandelion Root

Dandelion usually refers specifically to Taraxacum officinale. Taraxacum is a large genus of flowering plants in the family Asteraceae. Dandelion was prized historically for a variety of medicinal properties, and it contains a wide number of pharmacologically active compounds. Dandelion is also used as a herbal remedy in Europe, North America and China, to treat infections, bile and liver problems, and as a diuretic.

Dandelion root can be used as a sedative, anti-SNS or hypnotic in the instant invention.

### Catnip Leaf

Nepeta cataria, commonly known as catnip, catswort, or catmint, is a species of the genus Nepeta in the Lamiaceae family. The plant terpenoid nepetalactone is the main chemical constituent of the essential oil of Nepeta cataria. Nepetalactone can be extracted from catnip by steam distillation. Catnip has a history of medicinal use for a variety of ailments. The plant has been consumed as a tea, juice, tincture, infusion or poultice, and has also been smoked.

Catnip leaf can be used as a sedative in the instant invention.

### California Poppy

Eschscholzia californica (California poppy, golden poppy, California sunlight, cup of gold) is a species of flowering plant in the family Papaveraceae.

California poppy leaves were used medicinally by Native Americans, and the pollen was used cosmetically. The seeds are used in cooking. An aqueous extract of the plant has sedative and anxiolytic action. The extract acts as a mild sedative when smoked, and the effect is far milder than that of opium. California poppy is believed to contain a different class of alkaloids, and the extract appears to possess an affinity for the benzodiazepine receptor. The extract induced peripheral analgesic effects in mice but did not possess antidepressant, neuroleptic or antihistaminic effects. Thus California poppy can be used as a sedative in the instant invention.

### Plantain Seed Husks

Plantago is a genus of about 200 species of small, inconspicuous plants commonly called plantains. Plantain seed husks expand and become mucilaginous when wet, especially those of P. psyllium, which is used in common over-the-counter bulk laxative and fiber supplement products such as Metamucil. P. psyllium seed is useful for constipation, irritable bowel syndrome, dietary fiber supplementation, and diverticular disease. Psyllium supplements are typically used in powder form, along with adequate amounts of fluids. There are a number of psyllium products used for constipation. The usual dose is about 3.5 grams twice a day.

Mucilage from Desert Indianwheat (Plantago ovata) is obtained by grinding off the husk. This mucilage, also known as Psyllium, is commonly sold as Isabgol, a laxative which is used to control irregular bowel syndrome and constipation. It has been used as an indigenous Ayurvedic and Unani medicine for a whole range of bowel problems.

Plantain leaves have been given as a tea for cold and cough at 3 to 6 g/day, and can be used similarly.

### Senna

Senna (such as Cassia acutifolia and augustifolia) is a large genus of flowering plants in the legume family Fabaceae, and the subfamily Caesalpinioideae.

Sennas have played a major role in herbalism and folk medicine. Alexandrian senna (S. alexandrina) has long been traded commercially. Senna glycosides, or sennosides, are used in modern medicine as laxatives. Senna drugs contain the dried leaves of S. alexandrina. The glycosides increase gastric fluid secretion and bowel motility, producing laxative action. Senna preparations are available in powders, granules, tablets, oral infusions, and syrups. It is also available in combination with the dietary fiber psyllium to add bulk to the bowel contents. Several Senna species are used as herbal remedies in Nigeria to treat various conditions, including constipation, fungal skin infections, and hemorrhoids.

Usual adult dose for bowel preparation in oral form can be syrup, at a dose of 8.8 mg/5 mL: 10 to 15 mL (17.6 to 26.4 mg) orally once a day, not to exceed 15 mL (26.4 mg) twice a day; 176 mg/5 mL: 10 to 15 mL (352 to 528 mg) orally once a day, not to exceed 15 mL (528 mg) twice a day. Or it can be tablets, at 8.6 mg: 2 tablets (17.2 mg) orally at bedtime, not to exceed 4 tablets (34.4 mg) twice a day; 17.2 mg: 1 tablet (17.2 mg) orally at bedtime, not to exceed 2 tablets (34.4 mg) twice a day; 25 mg: 2 tablets (50 mg) orally once or twice a day; 17 mg: 1 tablet (17 mg) orally, not to exceed 2 tablets (34 mg) twice a day; 15 mg: 2 tablets (30 mg) orally once or twice a day. For pediatric uses, reduce doses accordingly.

Usual adult dose for constipation in oral form can be syrup, at a dose of 8.8 mg/5 mL: 10 to 15 mL (17.6 to 26.4 mg) orally once a day, not to exceed 15 mL (26.4 mg) twice a day; 176 mg/5 mL: 10 to 15 mL (352 to 528 mg) orally once a day, not to exceed 15 mL (528 mg) twice a day. Or it can be tablets of 8.6 mg: 2 tablets (17.2 mg) orally at bedtime, not to exceed 4 tablets (34.4 mg) twice a day; 17.2 mg: 1 tablet (17.2 mg) orally at bedtime, not to exceed 2 tablets (34.4 mg) twice a day; 25 mg: 2 tablets (50 mg) orally once or twice a day; 17 mg: 1 tablet (17 mg) orally, not to exceed 2 tablets (34 mg) twice a day; 15 mg: 2 tablets (30 mg) orally once or twice a day. For pediatric uses, reduce doses accordingly.

Additional additives contemplated for use herein may include various natural, health-enhancing additives associated with improved healing and well-being, such as vitamins, minerals and herbal and other plant and animal products. For example, vitamins and minerals, such as the B-complex vitamins, vitamins A and C and zinc, as well as others well known to those of skill in the art, are likewise considered useful herein for supplementing the present beverage composition and conferring additional benefits thereto.

In addition to the active ingredients above, the beverage composition is preferably also palatable to the patient, so as to encourage compliance with the requirements that the entire amount of the beverage be consumed prior to surgery.

Numerous possible additives are available and known in the art, any and all of which may be included in the subject beverage to increase palatability without significantly affecting the intended purpose of the subject beverages. For example, flavorings, such as chamomile, licorice, peppermint, ginger, comfrey, citric acid, coffee, tea flavors, raspberry, lemon (e.g., lemon peel granual), and similar flavors may be added to the beverage to enhance its acceptability. However, chamomile, peppermint, and/or comfrey may be omitted as flavor if one or more of such ingredients are not compatible with the active ingredient(s) of a subject beverage.

In addition to flavoring, it may be desirable to add caffeine to the beverage composition; in particular for those patients that are regular caffeine consumers, as such patients frequently suffer from headaches when deprived of their normal caffeine ingestion. However, some recent studies have suggested that caffeine may have adverse effects on the acidity of stomach contents, and furthermore, Food and Drug Administration (FDA) regulations concerning the addition of caffeine to beverages may be prohibitive. Therefore, the amounts of caffeine used in the presently contemplated beverage composition, if any, should be very carefully monitored.

The subject beverages may contain no caffeine or caffeine-like substances.

Numerous other additives may also be included within the subject beverage compositions. For example, an aspiration prophylaxis may be included in the beverage composition in order to further reduce the risk of acid aspiration during induction of or emergence from anesthesia and/or sedation. Aspiration prophylaxis, such as the class of compounds defined as selective serotonin 5HT3 receptor antagonists, are known to those of skill in the art. Examples of aspiration prophylaxis include such compounds as ranitidine, ondansetron (Zofran, Glaxo Wellcome, Research Triangle Park, N.C.) and metoclopramide. Other histamine antagonists, likewise known to those of skill in the art, may also be included within the beverage, as well as known antacids, such as 0.3 M sodium citrate or Bicitra (Baker Norton Pharmaceuticals, Inc., Miami, Fla.), a urinary alkalinizing drug that contains about 0.3 M sodium citrate.

It will be appreciated by those of skill in the art that there will be certain contra-indications for use of this pre-operative beverage composition. For example, diabetic patients, pregnant women, trauma victims, the narcotized, and the massively obese patient, as well as any other patient having either a mechanical or neurological basis for retarded gastric emptying should probably not use this beverage. However, for the vast majority of otherwise healthy patients the pre-operative beverage composition contemplated and described herein, may be safely administered with expectations of reducing their risk of acid aspiration during anesthesia / sedation and of improving the general well-being of the patient both pre-and post-surgery.

### FORMULARY EXAMPLES

Using organically grown herbs, or the concentrated extracts of the highest quality herbs, the following recipe could be derived with attention paid to the types and concentration of herbs included, the amount of water to bring the volume to a total of one liter in a sealed bottle. Certain of the herbs listed above have multiple properties, such as antiemesis and sedation. Certain have one or both of those properties and also have strong flavorant properties and colorant properties. The variation of these herbal elements allows the beverages to naturally be of differing attractive flavors, and odors as well as being actively sedating, forward propulsive gastrointestinally and antiemetic.

One beverage is designed to be a tasteful, pleasantly appearing aqueous mixture of herbs that have properties of anti-nausea, well-being, and mildly sedating, or calming.

Another beverage is designed to be a tasteful, pleasantly appearing aqueous mixture of herbs that have properties consistent with stress reduction, mild sedation, positive effects on GI tract peristalsis, and thus emptying the upper GI tract and thereby decreasing the chances of regurgitation of GI contents.

Some or all of the beverages described herein are to have a plurality of effects, which could be any combination of anti-nausea, antiemetic, anti-stress, mildly calming, mildly sedating, pro-peristaltic, anti-gastroparetic, and decreasing the activity of the sympathetic nervous system.

The following formulation examples are provided for the sole purpose of illustrating certain specific embodiments of the present invention. They are, however, in no way limiting to the general scope of the invention.

On the other hand, however, each of the specific formulations described below constitute an illustrative embodiment of the present invention.

### Example 1

A stable soluble ingestible herbal brew was prepared by blending together: organic Poppy Flowers (Eschscholzia californica) 4 grams (sedative), organic Orange peel Powder 2.5 grams (flavorant), Cardamom seed (anti-GERD and anti-Nausea/Vomit), and 500 mL water.

The brew was prepared by bringing water to a consistent temperature of about 212° F. degrees, followed by lowering and raising the combination of the indicated herbs into and out of the water (e.g., as in lowering and raising a tea bag) in order to ensure complete saturation, circulation, and absorption of the herbs, for about four minutes. The result was an odorless herbal beverage blend of violet color, pleasing taste, and a relatively neutral pH level of 6.

This brew was ingested by 12 healthy volunteer test subjects. Within 15 minutes of ingestion, each test subject became calm, relaxed, with a sense of well-being.

In cases where additional flavor or scent is desired, about 2.5 grams of Organic Spearmint may be added, which results in a slightly elevated yet still relatively neutral pH of 6.5. In addition, 2.5 grams of Organic Honey can be added as a flavorant with no resulting change in pH level.

### Example 2

Another soluble ingestible herbal brew was prepared by blending together: organic Silk Flower (Mimosa flowers or Silk Tree) 4 grams (sedative), organic Raspberry Leaf 4 grams (flavorant and anti-Nausea/Vomit), organic Ginger Root Extract 2 cc (anti-NV and anti-motion sickness), Tangerine Essential Oil 2 cc (flavorant and properistalsis), and 750 mL water.

The brew was prepared by bringing water to a consistent temperature of about 212° F. degrees, followed by lowering and raising the combination of the indicated herbs into and out of the water (e.g., as in lowering and raising a tea bag) in order to ensure complete saturation, circulation, and absorption of the herbs, for about four minutes. The result was a pleasantly scented, golden honey colored herbal beverage blend, with a pleasing taste and a relatively neutral pH level of 6.5.

This brew was ingested by 10 healthy volunteer test subjects. Within 30 minutes of ingestion, each test subject became calm, relaxed, and experienced a mellow feeling.

### Example 3

Another soluble ingestible herbal brew was prepared by blending together: organic Linden Leaf and Flower 4 grams (sedative and anti-SNS), organic Whole Jasmine Flowers 4 grams (sedative and anti-SNS), organic Lemongrass C/S 2 grams (sedative and flavorant), organic Plantain Leaf 2 grams (anti-GERD), organic Red Clover Blossom Extract 2 cc (anti-NV), and 500 mL water.

The brew was prepared by bringing water to a consistent temperature of about 212° F. degrees, followed by lowering and raising the combination of the indicated herbs into and out of the water (e.g., as in lowering and raising a tea bag) in order to ensure complete saturation, circulation, and absorption of the herbs for about four minutes. The result was a slightly citrus scented, clear colored, pleasant tasting herbal beverage with a relatively neutral pH level of 6.

12 healthy volunteer test subjects ingested this brew. Within 15 minutes of ingestion, each test subject became calm, relaxed, with a sense of well-being.

### Example 4

Another soluble ingestible herbal brew was prepared by blending together: organic Whole Jasmine Flowers 4 grams (sedative and anti-SNS), organic Poppy Flower 4 grams (sedative and flavorant and anti-SNS), organic Spearmint 2.5 grams (flavorant and anti-SNS and anti-Nausea/Vomit (or “anti-N/V” in short)), organic Passion Flower Leaf Powder 1 teaspoon (sedative and anti-SNS), and 750 mL water.

The brew was prepared by bringing water to a consistent temperature of about 212° F. degrees, followed by first placing the organic Passion Flower Leaf Powder into the water, which fully dispersed in one minute, then by lowering and raising the combination of the other indicated herbs into and out of the water in order to ensure complete saturation, circulation, and absorption of the herbs for about four minutes. The result was a slightly mint scented, violet colored, pleasant tasting herbal beverage with a relatively neutral pH level of 6.

This brew was ingested by 15 healthy volunteer test subjects. Within 15 minutes of ingestion, each test subject became calm, relaxed, with a sense of well-being.

### Example 5

Another soluble ingestible herbal brew was prepared by blending together: organic Calendula Flowers Whole 4 grams (sedative), organic Chamomile Flower Powder 4 grams (sedative and anti-SNS), organic Lemon Balm Leaf Powder 1 teaspoon (sedative and flavorant and anti-SNS), organic Cinnamon Granules 1 teaspoon (flavorant and properistalsis and anti-GERD), and 750 mL of water.

The brew was prepared by bringing water to a consistent temperature of about 212° F. degrees, followed by first placing the organic Lemon Balm Leaf Powder and organic Cinnamon Granules into the water, which fully dispersed in about 60-90 seconds, then by lowering and raising of the combination of the remaining indicated herbs into and out of the water in order to ensure complete saturation, circulation, and absorption of the herbs for about four minutes. The result was a slightly lemon scented, pale lemon colored, pleasant tasting herbal beverage with a relatively neutral pH level of 6.

This brew was ingested by 10 healthy volunteer test subjects. Within 15 minutes of ingestion, each test subject became calm, relaxed, with a sense of well-being.

### Example 6

Another soluble ingestible herbal brew was prepared by blending together: organic Hops Flowers Powder 1 teaspoon (sedative and anti-SNS and anti-GERD), organic California Poppy Herb C/S 4 grams (sedative and flavorant and anti-SNS), organic Meadowsweet Herb C/S 4 grams (sedative and flavorant and anti-N/V), Raspberry Leaf 2 grams (flavorant), Honey 1 teaspoon, and 750 mL water.

The brew was prepared by bringing water to a consistent temperature of about 212° F. degrees, followed by first placing the organic Hops Flowers Powder into the water, which fully dispersed in about one minute, then by lowering and raising of the combination of the remaining indicated herbs into and out of the water in order to ensure complete saturation, circulation, and absorption of the herbs for about four minutes. The result was a non-scented, slightly rose colored, pleasant tasting herbal beverage with a relatively neutral pH level of 6.2.

This brew was ingested by 12 healthy volunteer test subjects. Within 15 minutes of ingestion, each test subject became calm, relaxed, with a sense of well-being.

### Example 7

Another soluble ingestible herbal brew was prepared by blending together: organic Poppy Flowers 4 grams (sedative), organic Jasmine Flowers Whole 4 grams (sedative and anti-SNS), organic Silk Tree Flowers 4 grams (sedative), organic Spearmint Leaves 2 grams (flavorant and anti-SNS and anti-N/V), organic Caraway Seed Powder 2 grams (flavorant and anti-SNS), and 750 mL water.

The brew was prepared by bringing water to a consistent temperature of about 212° F. degrees, followed by first placing the organic Caraway Seed Powder into the water, which fully dispersed in approximately 90 seconds, then by lowering and raising the combination of the remaining indicated herbs into and out of the water in order to ensure complete saturation, circulation, and absorption of the herbs for about four minutes. The result was a slightly Caraway scented, light amber colored, pleasant tasting herbal beverage with a relatively neutral pH level of 6.5.

This brew was ingested by 10 healthy volunteer test subjects. Within 15 minutes of ingestion, each test subject became calm, relaxed, with a sense of well-being.

### Example 8

Another soluble ingestible herbal brew was prepared by blending together: organic Silk Tree Flowers 4 grams (sedative), organic Lavender Flowers 4 grams (sedative and anti-SNS), organic Dandelion Root Extract 2 grams (sedative and anti-SNS), organic Lemon Peel Granules 1 teaspoon (flavorant), and 750 mL water.

The brew was prepared by bringing water to a consistent temperature of about 212° F. degrees, followed by first placing the organic Lemon Peel Granules into the water, which fully dispersed in about one minute, then by lowering and raising the combination of the remaining indicated herbs into and out of the water in order to ensure complete saturation, circulation, and absorption of the herbs for about four minutes. The result was a lightly citrus scented, light amber colored, pleasant tasting herbal beverage with a relatively neutral pH level of 6.

This brew was ingested by 12 healthy volunteer test subjects. Within 15 minutes of ingestion, each test subject became calm, relaxed, with a sense of well-being.

### Example 9

Another soluble ingestible herbal brew was prepared by blending together: organic Silk Tree Flowers 4 grams (sedative), organic Hyssop Herb 4 grams (sedative and anti-SNS and anti-GERD), organic Passion Flower Leaf Powder 2 grams (sedative and anti-SNS), organic Orange Peel Granules 1 teaspoon (flavorant), Honey 1 teaspoon, and 750 mL water.

The brew was prepared by bringing water to a consistent temperature of about 212° F. degrees, followed by first placing the organic Passion Flower Leaf Powder and organic Orange Peel Granules into the water, which fully dispersed in about 60 to 90 seconds, then by lowering and raising of the combination of the remaining indicated herbs into and out of the water in order to ensure complete saturation, circulation, and absorption of the herbs for about four minutes. The result was a lightly sweet scented, medium amber colored, pleasant tasting herbal beverage with a relatively neutral pH level of 6.2.

This brew was ingested by 15 healthy volunteer test subjects. Within 15 minutes of ingestion, each test subject became calm, relaxed, and a mellow feeling.

1. An aqueous beverage composition having a substantially neutral pH and having substantially no calories from carbohydrates, wherein the beverage, upon consumption by or administration to a subject, hydrates the subject towards euvolemia.

2. An aqueous beverage composition having a substantially neutral pH and having substantially no calories from carbohydrates, wherein the beverage, upon consumption by or administration to a subject, induces forward propulsion of the gastrointestinal (GI) tract of the subject.

3. An aqueous beverage composition having a substantially neutral pH and having substantially no calories from carbohydrates, wherein the beverage, upon consumption by or administration to a subject, induces a feeling of well-being and decreased anxiety in the subject.

4. An aqueous beverage composition having a substantially neutral pH and having substantially no calories from carbohydrates, wherein the beverage, upon consumption by or administration to a subject, decreases sympathetic nervous system (SNS) function or activity in the subject, and/or increases parasympathetic nervous system (PSNS) function/activity in the subject.

5. An aqueous beverage composition having a substantially neutral pH and having substantially no calories from carbohydrates, wherein the beverage, upon consumption by or administration to a pre-operative patient, decreases the risk of aspiration pneumonitis while simultaneously improving the well-being of the patient.

6. The aqueous beverage composition of claim 1, wherein the subject is undergoing pre-operative preparation, and the beverage, upon consumption by or administration to the subject, decreases the risk of aspiration pneumonitis while simultaneously improving the well-being of the subject.

7. The aqueous beverage composition of claim 1, wherein the subject is about to experience a stressful condition or an important event.

8. The aqueous beverage composition of claim 7, wherein the stressful condition and/or an important event include: a public speech or presentation, a test/exam, a social event (wedding ceremony, attending sports event, etc.), a non-surgical procedure (e.g., a dental procedure, cardiac catheterization, or GI endoscopy).

9. The aqueous beverage composition of claim 1, wherein the neutral pH is about pH 5.5-8, about 6-8, about 5.5-6.5, about 6.5-7.5, about 6, or about 7.

10. The aqueous beverage composition of claim 9, wherein the beverage maintains substantially neutral pH in case of aspiration of stomach contents.

11. The aqueous beverage composition of claim 1, wherein the total calories is no more than about 150 Cal/serving, no more than about 100 Cal/serving, no more than about 50 Cal/serving, or no more than about 20 Cal/serving.

12. The aqueous beverage composition of claim 1, wherein the beverage is non-particulate.

13. The aqueous beverage composition of claim 1, wherein the beverage is non-particulate in case of aspiration of stomach contents.

14. The aqueous beverage composition of claim 1, wherein the beverage has a total salt concentration equivalent to or within 10% of that of the intravascular fluid in said subject or patient.

15. The aqueous beverage composition of claim 14, wherein the beverage has a total salt concentration of about 140 mEQ.

16. The aqueous beverage composition of claim 1, wherein the beverage comprises a first herbal composition that induces forward propulsion of the gastrointestinal (GI) tract contents with forward peristalsis of the GI tract of the subject.

17. The aqueous beverage composition of claim 16, wherein said first herbal composition comprises: Clove bud, meadow sweet, cardamom, peppermint, fennel, cinnamon, goldthread root, hops, cannabis, caraway, tangerine peel, alfalfa herb, anise seed, Hysop, Chamomile flower, ginger, slippery elm, wild corn poppy, Raspberry leaf, red clover, or spearmint, plantain, or a mixture of two or more thereof.

18. The aqueous beverage composition of claim 17, wherein each constituent of said first herbal composition is in substantially equal concentrations (w/w), and preferably the aqueous beverage composition is q.s. to one liter total volume.

19. The aqueous beverage composition of claim 1, wherein the beverage comprises a second herbal composition that induces a feeling of well-being and decreased anxiety in the subject.

20. The aqueous beverage composition of claim 19, wherein said second herbal composition comprises: chamomile, lemon balm, lemon verbena, lemon grass, mullein flower, basil leaf, Hysop leaf, hops, linden, passion flower, American valerian root or valerian, mimosa flower, bitter orange leaf or flowers, jasmine, lavender, Lotus corniculatus, calendula, dandelion root, catnip leaf, or California poppy, meadow sweet, or a mixture of two or more thereof.

21. The aqueous beverage composition of claim 20, wherein each constituents of said second herbal composition is in substantially equal concentrations (w/w), and preferably the aqueous beverage composition is q.s. to one liter total volume.

22. The aqueous beverage composition of claim 1, wherein the beverage comprises a third herbal composition that decreases sympathetic nervous system (SNS) function or activity in the subject, that is antiemetic, antacid, pro-peristaltic, or mildly sedative; and/or that enhances well-being.

23. The aqueous beverage composition of claim 22, wherein said third herbal composition comprises: hops, valerian, chamomile, passion flower, lemon balm, linden, California poppy, spearmint, tangerine, or a mixture of two or more thereof.

24. The aqueous beverage composition of claim 23, wherein each constituent of said third herbal composition is in substantially equal concentrations (w/w), and preferably the aqueous beverage composition is q.s. to one liter total volume.

25. The aqueous beverage composition of claim 1, wherein the beverage comprises a flavorant.

26. The aqueous beverage composition of claim 25, wherein the flavorant comprises: orange peel, tangerine, lemongrass, poppy, spearmint, lemon balm, cinnamon, California poppy, meadowsweet, Raspberry Leaf, Caraway, or lemon.

27. The aqueous beverage composition of claim 16, wherein said first, second, or third herbal composition is a US FDA-approved composition for human administration.

28. The aqueous beverage composition of claim 27 wherein the beverage reduces fears, decreases pre-operative jitters or butterfly stomachs, reduced acidity of the stomach contents, reduces a feeling of nausea with an empty stomach, or a feeling of pre-operative parchedness, and/or reduces pre-operative headaches from stress and migraines.

29. The aqueous beverage composition of claim 27 wherein the ratio or amount of the second herbal composition is adjusted to induce sleepiness in the subject, and wherein the subject has insomnia and GERD (gastroesophageal reflux disease).

30. A concentrate or an essentially dry mixture, suitable for use in forming the aqueous beverage of claim 1, comprising an admixture of materials in amounts and ratios such that, when constituted with water, said aqueous beverage is formed.

31. A method of preparing a patient for induction of anesthesia for surgery, the method comprising instructing the patient to ingest, beginning on midnight prior to a day said surgery or anesthesia is scheduled for the patient, an aqueous beverage composition of claim 1 ad lib.

32. The method of claim 31, further comprising instructing the patient to refrain from ingesting the aqueous beverage composition about 2-3 hours before said induction.

33. A method of preparing a patient for induction of anesthesia for surgery, the method comprising causing the patient to ingest, beginning on midnight prior to a day said surgery or anesthesia is scheduled for the patient, an aqueous beverage composition of claim 1 ad lib.

34. The method of claim 33, further comprising causing the patient to refrain from ingesting the aqueous beverage composition about 2-3 hours before said induction.

35. A method of preparing a subject for experiencing a stressful condition or participating in a social event, the method comprising instructing the subject to ingest, prior to experiencing the stressful condition or participating in the social event, an aqueous beverage composition of claim 1 ad lib.
